(19) United States
(12) Patent Application Publication
(10) Pub. No.: US 2026/0324955 A1
(43) Pub. Date: Oct. 22, 2026

(54) ENDOSCOPIC SURGICAL SYSTEMS, DEVICES, AND METHODS

(71) Applicant: OLYMPUS CORPORATION, Tokyo (JP)

(72) Inventors: Michael BLOM, Cambridge (GB); Alex NORMAN, Cambridge (GB); Andrew FORBES, Cambridge (GB); Wayne GIMBLETT, Cambridge (GB); Gary STACEY, Cambridge (GB); Ryohei OGAWA, Hachioji-shi (JP); Ryoji HYODO, Hachioji-shi (JP); Yuta FUJII, Hachioji-shi (JP); Yuji SAKAI, Hachioji-shi (JP); Takeshi ARAI, Hachioji-shi (JP); Naoya HATAKEYAMA, Hachioji-shi (JP); Masato NARISAWA, Hachioji-shi (JP); Shotaro TAKEMOTO, Hachioji-shi (JP); Yuhei TAKATA, Hachioji-shi (JP); Masahiro FUJII, Hachioji-shi (JP); Ryu KUBO, Hachioji-shi (JP); Shintaro INOUE, Westborough, MA (US)

(21) Appl. No.: 19/570,066

(22) Filed: Mar. 17, 2026

Related U.S. Application Data
(60) Provisional application No. 63/772,981, filed on Mar. 17, 2025.
(60) Provisional application No. 63/795,879, filed on Apr. 28, 2025.

(51) Int. Cl. A61B 1/005; A61B 34/30; A61B 34/37

(52) U.S. Cl. CPC A61B 1/0053; A61B 34/37; A61B 2034/301

(57) ABSTRACT

Disclosed herein are systems, devices, and methods for endoscopic surgery. Such a systems may include and endoscope including a distal end and a treatment manipulator at the distal end of the endoscope where the treatment manipulator includes a based, one or more arms each having a proximal end coupled to the base, and a plurality of artificial muscles where a proximal end of each muscle is coupled to the base and distal end of each muscle is coupled a distal end of the one of the one or more arms via a cable. An artificial muscle may comprise a body including a lumen, one or more elastomer layers, and a braided layer around the lumen, a port at least partially within the lumen that includes and engagement feature and/or locking feature, and crimp coupled to an end portion of the body and the port to seal the body to the port via the engagement feature and/or locking feature.

### INCORPORATION BY REFERENCE

This application claims priority to U.S. Provisional Application No. 63/772,981 filed on Mar. 17, 2025, and U.S. Provisional Application No. 63/795,879 filed on Apr. 28, 2025, each of which is hereby incorporated by reference in its entirety.

### TECHNICAL FIELD

This invention relates generally to systems, devices, and methods for endoscopic surgery.

### BACKGROUND

Endoscopic surgical systems, including robotic platforms, allow surgeons to perform minimally invasive procedures by introducing an endoscope with one or more manipulators or end effectors into a patient's body. These systems generally provide visualization via a camera at or near the distal end of the endoscope while the manipulator(s) carry tools for grasping, cutting, suturing, or otherwise interacting with tissue. Although such systems have improved surgical dexterity and access, they still face limitations, particularly in the manipulator portion. For example, controlling fine motion of the manipulator or tools coupled thereto can be challenging due to constraints imposed by the narrow dimensions of an endoscopic shaft and by conventional actuation approaches, such as tendon or pull-wire systems, that may introduce reduced precision or limited range of motion. The compactness required for endoscopic instruments can also restrict joint articulation and effective tool reach, making it difficult to achieve smooth movement or consistent tool orientation during a procedure. Visualization can further be hindered when manipulators or tools obscure the field or when camera placement is constrained by the same limited space. Accordingly, additional systems and devices for endoscopic surgery are desirable.

### SUMMARY

Disclosed herein are systems, devices, and methods for endoscopic surgery. For instance, artificial muscles for actuating for a treatment manipulator of a surgical system are described herein. An artificial muscle may comprise a tubular elongate body comprising a first elastomer layer forming an elongate body lumen, a second elastomer layer around the first elastomer layer, and a braided layer between the first and the second elastomer layers. The artificial muscle may further comprise a port positioned at least partially within the elongate body lumen and comprising an engagement feature and a locking feature and a crimp coupled to a proximal portion of the elongate body and the port. The crimp may be configured to seal the elongate body to the port via the engagement feature and the locking feature.

In some variations, the elongate body lumen may be configured to receive a fluid to change a dimension of the artificial muscle. In some variations, the elongate body may be configured to contract when the fluid is pressurized. In some variations, the braided layer may constrain longitudinal expansion of the tubular elongate body. In some variations, the engagement feature may comprise one or more grooves. In some variations, the engagement feature may comprise a thread. In some variations, the engagement feature may be a first engagement feature and the crimp may comprise a second engagement feature configured to interlock with the first engagement feature through the elongate body. In some variations, the port may comprise a port lumen in fluid communication with the elongate body lumen.

In some variations, the port may comprise a port body and a flange. In some variations, the flange may be configured to receive a hydraulic input pipe. In some variations, the port body may be configured to be positioned within the elongate body lumen. In some variations, the port body may comprise the engagement feature. In some variations, the port body may comprise the locking feature, where the locking feature is proximal to the engagement feature. In some variations, the port body may comprise a port body lumen of a first diameter and the flange comprise a flange lumen of a second, different diameter. In some variations, the crimp may be received on a surface of the port body between the locking feature and the flange.

In some variations, the artificial muscle may further comprise an end cap positioned at least partially within the elongate body lumen and comprising a cap engagement feature and a cap locking feature. In some variations, the end cap may be solid. In some variations, the end cap may be positioned in an end portion of the elongate body lumen and the port is positioned in an opposite end portion of the elongate body lumen.

In some variations, the crimp may be positioned around the locking feature of the port. In some variations, the crimp may be configured to compress a portion of the elongate body against the engagement feature. In some variations, the crimp may be further configured to secure the elongate body to the locking feature. In some variations, the locking feature may be outside of the elongate body lumen. In some variations, the locking feature may comprise a protrusion. In some variations, one or both of the first elastomer layer or the second elastomer layer may comprise uncured silicon.

In some variations, a portion of the elongate body may be compressed between the engagement feature of the port and the crimp. In some variations, the portion may comprise the first elastomer layer and the braided layer. In some variations, the portion may include only the braided layer. In some variations, the first elastomer layer may be positioned around the locking feature. In some variations, a portion of the braided layer may be positioned around the locking feature. In some variations, a portion of the port within the elongate body lumen may increase in diameter along a length of the port within the elongate body. In some variations, the elongate body may further comprise a coating configured to reduce vapor permeability.

In some variations, an artificial muscle may comprise a tubular elongate body comprising a first elastomer layer forming an elongate body lumen, a second elastomer layer around the first elastomer layer, and a braided layer between the first and the second elastomer layers, a port positioned at least partially within the elongate body lumen and comprising one or more grooves to engage the elongate body and a circumferential protrusion to lock the port to the elongate body, and a crimp coupled to a proximal portion of the elongate body and the port, wherein the crimp is configured to seal the elongate body to the port via the one or more grooves and the circumferential protrusion.

Also described herein are endoscopic surgical systems. In some variations, an endoscopic surgical system may comprise comprising an insertion manipulator comprising a distal end and a treatment manipulator at least partially positioned in the distal end of the insertion manipulator. The treatment manipulator may comprise a base, an arm comprising a proximal end coupled to the base, and a plurality of artificial muscles. In some variations, a proximal end of each muscle may be coupled to the base and a distal end of each muscle is coupled to a distal end of the arm via a respective cable.

In some variations, the endoscopic surgical system may further comprise a camera coupled to the distal end of the insertion manipulator. In some variations, the system may be configured such that the plurality of artificial muscles is outside a field of view of the camera at the distal end of the treatment manipulator when a tool received in the treatment manipulator is in use. In some variations, the arm may comprise a lumen configured to receive a treatment (e.g., surgical) tool therethrough. In some variations, the plurality of artificial muscles may control a position of the treatment tool by manipulating the arm.

In some variations, the plurality of artificial muscles may be configured to bend a proximal portion of the arm. In some variations, a proximal portion of the arm may be configured to lock in a curved configuration when bent. In some variations, the arm may comprise a plurality of vertebrae configured to lock in the curved configuration to maintain the curve of the proximal portion of the arm. In some variations, the plurality of artificial muscles may be configured to bend a distal portion of the arm. In some variations, a proximal portion of the arm may be configured to lock in a curved configuration while the distal portion is bent.

Also described herein are endoscopes. The endoscopes described herein may be used alone or in combination with the treatment manipulators described herein. In some variations, an endoscope may comprise an insertion manipulator comprising a distal portion and a central portion adjacent to the distal portion. The distal portion may comprise a distal tip and a pulley coupled to the distal tip. The endoscope may further comprise a cable anchored in the distal portion and coupled to the pulley, where the pulley increases a force applied by the cable to the distal portion when tension is applied to a proximal end of the cable.

In some variations, the force applied by the cable to the distal portion may be greater than the force applied by the cable to the central portion. In some variations, the cable may be radially offset from a longitudinal central axis of the insertion manipulator. In some variations, the force applied by cable may bend the endoscope about 180 degrees or more.

In some variations, an endoscope may comprise an insertion manipulator comprising a distal portion and a central portion adjacent to the distal portion, the distal portion comprising a distal tip and a pulley coupled to the distal tip and a cable coupled to the pulley. In some variations, the cable may comprise a first portion of the cable that extends from an anchor in the distal portion to the pulley and a second portion of the cable extends from the pulley to a proximal end of the insertion manipulator where the anchor and pulley increase a force applied by the cable to the distal portion when tension is applied a proximal end of the cable.

Also described herein are endoscopic surgical systems. In some variations, an endoscopic surgical system may comprise an insertion manipulator comprising a camera lumen and a recess at a distal end of the camera lumen where the recess comprises a larger cross-sectional dimension than a cross-sectional dimension of the camera lumen and a scope. The scope may comprise a housing configured to be received within the recess, a camera within the housing, and an elongate body coupled to the housing where the elongate body is slidably positioned in the camera lumen to adjust an axial position of the scope.

In some variations, the scope may be hermetically sealed. In some variations, the scope may further comprise a fiber optic cable within the elongate body. In some variations, the scope may further comprise an actuator. In some variations, the actuator may comprise a living hinge. In some variations, an angle of the housing may be adjustable by about 45 degrees or less relative to the insertion manipulator. In some variations, a distal end of the housing may deflect about 5 mm or less. In some variations, the camera lumen may comprise a seal configured to receive the elongate body of the scope. In some variations, the system may be configured for the scope to be inserted from a distal end of the insertion manipulator.

Also described herein are methods of using endoscopic surgical systems. In some variations, a method of using an endoscopic surgical system may comprise inserting a scope into a camera lumen of an insertion manipulator from a distal end of the insertion manipulator such that an elongate body of the scope is positioned within the camera lumen, inserting the insertion manipulator into a patient for a surgical procedure, and withdrawing the scope from the distal end of camera lumen by sliding the elongate body toward and out of the distal end of the insertion manipulator.

In some variations, the method may further comprise inserting a housing of the scope into a recess in the distal end of the insertion manipulator after the elongate body is inserted in the camera lumen. In some variations, the method may further comprise axially adjusting a position of the scope by sliding the scope within the camera lumen during the surgical procedure. In some variations, the scope may be hermetically sealed.

### BRIEF DESCRIPTION OF THE DRAWINGS

FIG. 1 depicts an illustrative variation of an exemplary endoscope system.

FIG. 2 depicts an illustrative variation of an insertion manipulator in a large intestine.

FIG. 3 depicts a perspective view of an exemplary insertion section of an insertion manipulator and an exemplary treatment manipulator.

FIG. 4 depicts a front view of a distal end portion of an exemplary insertion manipulator.

FIG. 5 depicts a cross-sectional view of a distal portion of an exemplary insertion manipulator.

FIG. 6A depicts a cross-sectional view of a distal portion of another exemplary insertion manipulator. FIG. 6B depicts an illustrative variation of simplified cross-sectional view of the insertion manipulator of FIG. 6A.

FIG. 7 depicts an illustrative variation of an insertion section and a bending wire of an exemplary insertion manipulator.

FIG. 8 depicts an illustrative variation of a distal portion of an insertion manipulator including a pulley.

FIG. 9 depicts illustrative variations of a distal portion of an insertion manipulator in varying curved configurations.

FIG. 10 depicts an exemplary outer sheath of an insertion manipulator.

FIG. 11 depicts another exemplary support of a bending section of an insertion section.

FIG. 12 depicts an exemplary treatment manipulator.

FIG. 13A depicts a perspective view of an exemplary artificial muscle. FIG. 13B depicts a perspective cross-sectional view of the exemplary artificial muscle of FIG. 13A.

FIG. 14 depicts a cross-sectional view of an end portion of an exemplary artificial muscle.

FIG. 15 depicts a cross-sectional view of and end portion of an exemplary artificial muscle including a port with a flared body.

FIG. 16A a perspective view of another exemplary artificial muscle. FIG. 16B depicts a perspective cross-sectional view of the artificial muscle of FIG. 16A.

FIG. 17 depicts a cross-sectional view of an end portion of an exemplary artificial muscle including a braided layer.

FIG. 18A depicts a perspective cross-sectional view of an exemplary artificial muscle including an elastomer layer molded to a port. FIG. 18B depicts a perspective view of the exemplary artificial muscle of FIG. 18A.

FIG. 19 depicts a cross-sectional view of an exemplary artificial muscle including a whip.

FIG. 20A depicts a perspective view of an exemplary artificial muscle showing a first elastomer layer. FIG. 20B depicts a perspective view of the artificial muscle of FIG. 20A showing second elastomer layer.

FIG. 21 depicts a cross-sectional view of an exemplary insertion manipulator and an exemplary treatment manipulator.

FIG. 22 depicts an exemplary treatment manipulator including shoulder mounted artificial muscles.

FIG. 23 depicts an illustrative variation of the field of view of an imaging unit observing the treatment of manipulator of FIG. 22.

FIG. 24A depicts an exemplary treatment manipulator in a delivery configuration. FIG. 24B depicts the treatment manipulator of FIG. 24A with a proximal portion of the treatment manipulator in a curved configuration. FIG. 24C depicts the treatment manipulator of FIGS. 24A and 24B with the proximal portion and a distal portion of the treatment manipulator in curved configurations.

FIG. 25 depicts a cross-sectional view of an exemplary insertion manipulator and an exemplary treatment manipulator where artificial muscles are proximal to a distal end of the insertion manipulator.

FIG. 26 depicts a cross-sectional view of an exemplary endoscope system including an artificial muscle positioned in a distal portion of an insertion manipulator.

FIG. 27 depicts a cross-sectional view of an exemplary endoscope system including an artificial muscle positioned in a central portion of an insertion manipulator.

FIG. 28 depicts a cross-sectional view of an exemplary endoscope system including an artificial muscle positioned in a proximal portion of an insertion manipulator.

FIG. 29 depicts a cross-sectional view of an exemplary endoscope system including a plurality of artificial muscles positioned in series in an insertion manipulator.

FIG. 30 depicts an exemplary endoscope system including a scope.

FIG. 31A depicts a perspective view of an exemplary imaging unit. FIG. 31B depicts an exploded view of the imaging unit of FIG. 31A.

FIG. 32 depicts an illustrative variation of an imaging unit adjustable via a wire.

FIG. 33 depicts a side view of an exemplary adjustable imaging unit.

FIG. 34A depicts an exemplary endoscope system including a slidable scope and a camera lumen seal. FIG. 34B depicts the camera lumen seal of FIG. 34A.

### DETAILED DESCRIPTION

Generally, described herein are systems, devices, and methods for endoscopic surgery. Endoscopic procedures may be performed to permit access to internal anatomical structures through small incisions or natural orifices, thereby reducing patient trauma while enabling manipulation and visualization within confined spaces of the body. Such procedures may rely on compact manipulators and imaging units introduced through narrow working channels to perform diagnostic or therapeutic operations while minimizing disruption of surrounding tissue. The systems and devices described herein may provide improved dexterity, control, and visualization relative to conventional approaches by enabling more precise manipulation within restricted anatomical corridors, enhancing articulation without increasing shaft diameter, and reducing occlusion of the visual field. The systems and devices described herein, and in particular treatment manipulators as described herein, may facilitate smoother motion, expanded effective reach, and more stable or adaptable camera positioning by using artificial muscle actuation.

The systems and devices described herein may be used in a variety of endoscopic procedures throughout the alimentary tract. Such procedures may include diagnostic and therapeutic operations performed within the colon and rectum, such as polypectomy, endoscopic mucosal resection, endoscopic submucosal dissection, hemostasis, foreign-body retrieval, or stricture dilation. Additional applications may include procedures within the esophagus, stomach, or small intestine, including biopsy, lesion resection, stent placement, or visualization and treatment of bleeding sites. The systems described herein may also be used for observation or treatment in a hollow organ, such as those of the alimentary canal, where access is achieved through natural luminal pathways. An endoscope system as described herein may be introduced through natural orifices, including the mouth, anus, or other suitable access points, to reach targeted regions of the digestive tract while avoiding external incisions. These systems may therefore be compatible with standard gastrointestinal endoscopic workflows while providing enhanced manipulation and visualization capabilities as described herein.

Endoscope systems as described herein may generally comprise an insertion manipulator, a treatment manipulator, and a scope, and, in some embodiments, one or more drive units configured to actuate the manipulators. The insertion manipulator may extend in a longitudinal direction and may include one or more lumens, including a lumen sized to receive the treatment manipulator and a separate scope lumen configured to accommodate an imaging unit. The treatment manipulator may be at least partially positioned at or near the distal end of the insertion manipulator and may comprise a base, an arm having a proximal end coupled to the base, and a plurality of artificial muscles for actuating the arm and/or tools coupled thereto. In certain embodiments, the system may further comprise a first drive unit configured to drive and/or actuate the insertion manipulator and a second drive unit configured to drive and/or actuate the treatment manipulator, wherein the first drive unit may be disposed distal to the second drive unit and may be movable in the longitudinal direction to support advancement or retraction of the insertion manipulator.

Also described herein are methods of using the systems and devices described herein. Methods of use may include positioning and advancing an insertion manipulator through a natural orifice and actuating a portion of the insertion manipulator and/or treatment manipulator to perform an endoscopic surgical operation. In some variations, the method may include actuating one or more artificial muscles of the treatment manipulator to move, orient, and/or operate a surgical tool at a target site within the body.

### I. Systems and Devices

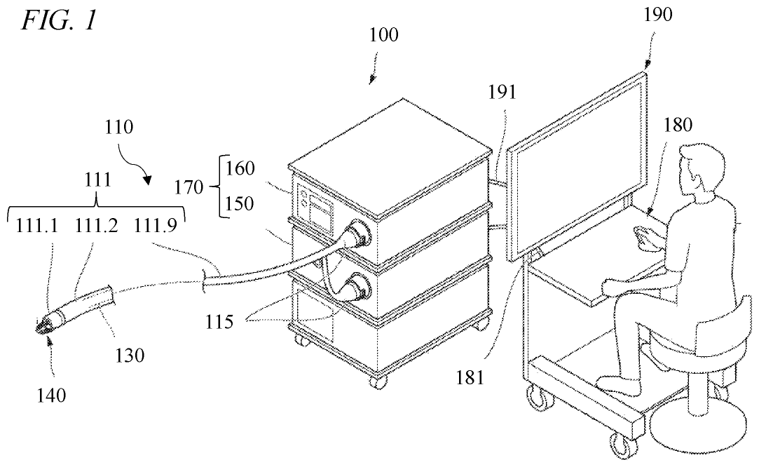

Described herein are endoscope systems for endoscopic surgery. FIG. 1 depicts an illustrative variation of an exemplary endoscope system 100. The endoscope system 100 is an example of a medical manipulator system and may include an insertion manipulator 110 configured to be inserted into a human body. The system may further include an endoscope, catheter, treatment tool, or other endoluminal device. These components may be mechanically or electrically driven and may be operated in coordination to perform observation and treatment within a patient.

The endoscope system 100 may be a medical system for observing and treating the inside of the body of a patient. The system may comprise an operating table configured to support the patient (e.g., while the patient is lying on the operating table). The endoscope system 100 may include an insertion manipulator 110, a treatment manipulator 140, a drive device 150, an image control device 160, an operation device 180, and a display device 190. The drive device 150 and the image control device 160 may constitute a control device 170 that controls the endoscope system 100.

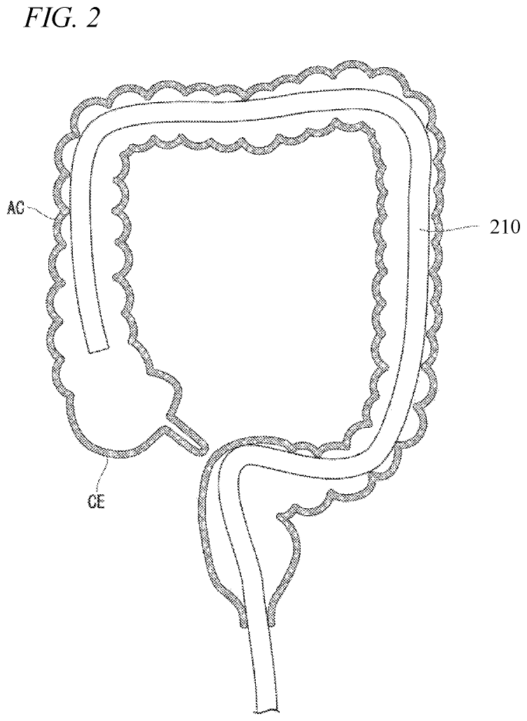

FIG. 2 depicts an insertion manipulator 210 positioned within a large intestine. The insertion manipulator 110 may be a device introduced into a lumen of a patient to observe and treat a lesion. The insertion manipulator 110 may be configured to be insertable and may be positioned, for example, in an ascending colon AC or a cecum CE of a large intestine as illustrated in FIG. 2. The insertion manipulator 110 may be detachably coupled to the drive device 150 and the video control device 160. An internal path 130 may be formed within the insertion manipulator 110. In the present description, a side of the insertion manipulator 110 inserted into a lumen of a patient is referred to as a “distal end side (distal side) A1,” and a side attached to the drive device 150 is referred to as a “proximal side (proximal side) A2.”

The treatment manipulator 140 may be a device inserted into a first channel tube 171 of the insertion manipulator 110, may protrude from a first opening 111a, and may be advanced into a lumen of a patient to treat the patient. An end effector (a treatment portion) that may treat a lesion may be disposed at the distal end of the treatment manipulator 140.

A scope (not shown) may be a unit that observes a lesion or other target tissue of interest and may be attached to the distal end portion 111.1. The scope may be bendably attached to protrude from the distal end portion 111.1 toward the distal end. The scope may include an imaging unit and an illumination unit.

The drive device 150 and the video control device 160 may constitute a control device 170 that controls the endoscope system 100. The control device 170 may further include peripherals such as a video printer. In some variations, the drive device 150 and the video control device 160 may be integrated into a unified device.

The drive device 150 may be detachably connected to the insertion manipulator 110 and the operation device 180. The drive device 150 may drive a motor built into the device on the basis of an operation input to the operation device 180 to actuate the insertion manipulator 110. The drive device 150 may also drive a pump or other fluid-handling mechanism on the basis of an operation input to the operation device 180 and may cause the insertion manipulator 110 to perform air supply, water supply, and suction.

The video control device 160 may be detachably connected to the insertion manipulator 110 and may acquire a captured image from the insertion manipulator 110. The video control device 160 may cause the display device 190 to display the captured image, as well as a GUI image or a CG image, to provide information to an operator.

The operation device 180 may be detachably connected to the drive device 150 via an operation cable 181. The operation device 180 may alternatively communicate with the drive device 150 in a wireless manner. An operator may actuate the insertion manipulator 110 by operating the operation device 180.

The display device 190 may be a device configured to display an image, such as an LCD. The display device 190 may be connected to the video control device 160 via a display cable 191.

The operation device 180 and the display device 190 may be mounted on a trolley. The trolley on which the operation device 180 and the display device 190 are mounted may also be referred to as a console.

The constituent devices of the endoscope system 100 are described below. In some variations, the endoscope system 100 or the entire insertion manipulator 110 may be considered to be an “endoscope.” Additional exemplary devices for an endoscope system that may be used with the systems, devices, and methods described herein are disclosed in U.S. Pat. Pub. No. 2025/0331941 filed Mar. 10, 2025, the contents of which is hereby incorporated by reference herein in its entirety.

### Insertion Manipulator

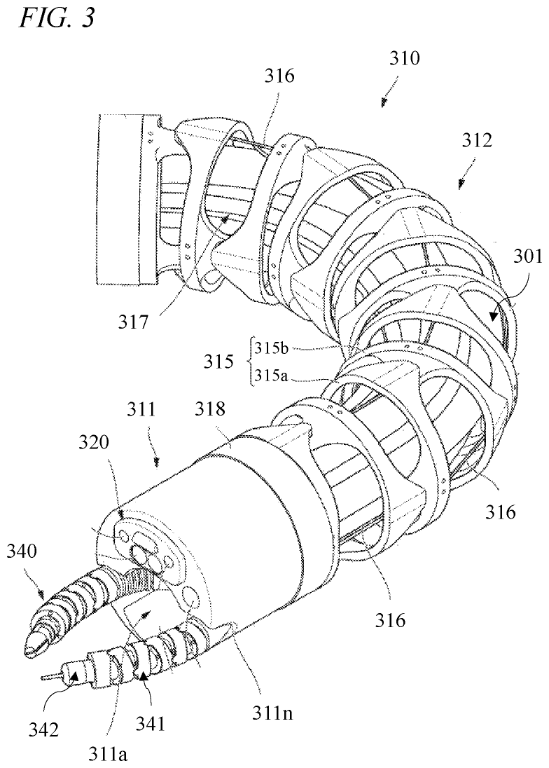

FIG. 3 depicts a perspective view of an exemplary insertion section 310 of an insertion manipulator. In addition to the insertion section 310, the insertion manipulator may include an attachment portion (e.g., 115 as shown in FIG. 1), a bending wire 316, and a built-in element 317. FIG. 3 further depicts a scope 320 within the insertion manipulator 310 and a treatment manipulator 340.

In the insertion manipulator, an internal path (e.g., a lumen) 301 may be formed so as to extend in a longitudinal direction (e.g., a longitudinal axis direction, an axial direction) of the insertion manipulator from the distal end of the insertion section 310 to the proximal end of the attachment portion (not shown). The bending wire 316 and the built-in element 317 may be positioned within the internal path 301.

The insertion section 310 may be a thin, elongated member configured to be inserted into a hollow organ or canal of a patient. The insertion section 310 may include a distal end portion 311, a bending portion 312, and a flexible portion (not shown, e.g., 111.9 of FIG. 1). The distal end portion 311, the bending portion 312, and the flexible portion may be sequentially arranged from the distal end toward the proximal end of the insertion manipulator. The insertion section 310 may include an outer sheath forming an outermost layer.

As shown in FIG. 1, the insertion section may comprise a flexible portion 111.9. The flexible portion may be a longitudinal, flexible tubular member. The flexible portion 111.9 may include an outer sheath that forms the outermost layer. A distal end of the flexible portion 111.9 may be connected to the bending portion 111.2. One or more cables, built-in elements, a first channel tube, a second channel tube, a scope channel, an imaging cable, a light guide, a water supply tube, a air supply tube, and/or a suction tube may be positioned within an internal path formed in the flexible portion 111.9.

The attachment portion 150 may be provided at a proximal end of the flexible portion 111.9, as illustrated in FIG. 1. The attachment portion 150 may be configured to couple the flexible portion 111.9 to the drive device 500 and the video control device 600. Other components and/or portions of the insertion manipulator are described in more detail below with respect to FIG. 3.

### Distal End Portion

In some variations, the distal end portion 311 of the insertion section may be rigid. To improve the maneuverability of the insertion section 310 within a patient, it may therefore be desirable to reduce a length of the distal end portion. For example, the length of the distal end portion of an insertion section of the devices described herein may be about 20 mm to about 40 mm, about 30 mm to about 40 mm, or about 20 mm to about 30 mm, including all subranges and values in any of the foregoing, such as, for example, about 20 mm, about 25 mm, about 28 mm, about 30 mm, about 35 mm or about 37 mm.

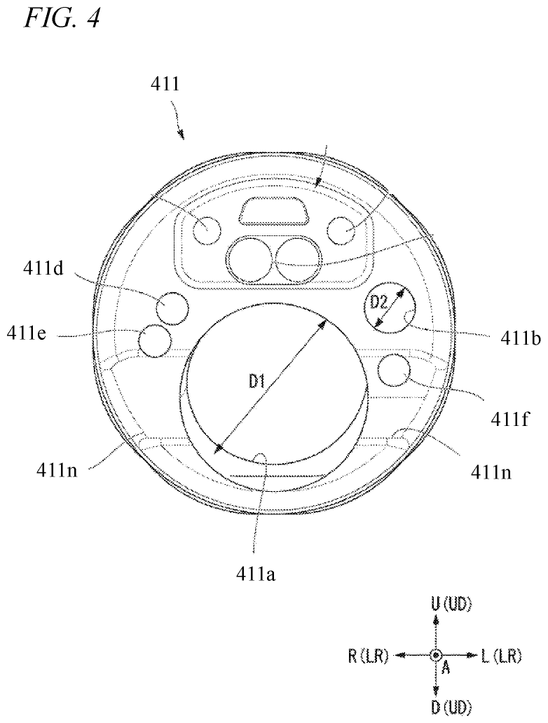

FIG. 4 is a front view of a distal end portion 411 of an insertion section of an insertion manipulator. The distal end portion 411 may be formed in a cylindrical shape and may include a first opening 411a, a second opening 411b, a water supply nozzle 411d, an air supply nozzle 41le, and a suction nozzle 411f. The first opening 411a, the second opening 411b, the water supply nozzle 411d, the air supply nozzle 41le, and the suction nozzle 411f may be formed on a distal end surface of the distal end portion 411. The first opening may be configured to receive a treatment manipulator. The first opening 411a may allow the treatment manipulator to extend from a treatment lumen of the insertion manipulator past a distal end surface of the treatment manipulator.

### Insertion Manipulator Channels

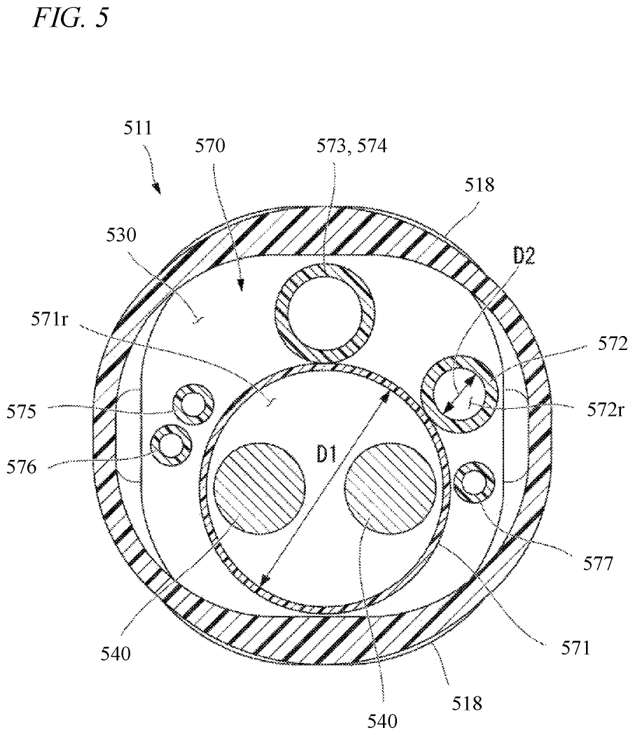

FIG. 5 depicts a cross-sectional view of a distal portion 511 of an insertion manipulator. In some variations, the insertion manipulator may comprise a built-in element 570 that forms one or more channels within the insertion manipulator to define one or more lumens. The built-in element 570 may reside in the internal path 530. In some variations, the built-in element may be formed integrally as part of the insertion manipulator. The built-in element 570 or the insertion manipulator itself may include a first channel tube 571, a second channel tube 572, an imaging cable 573, a light guide 574, a water supply tube 575, an air supply tube 576, a suction tube 577, and/or one or more scope operation wires (not shown). In FIG. 5, two treatment manipulators 540 that may be inserted into the first channel tube 571 are also illustrated.

Referring to both FIGS. 4 and 5, the first opening 411a may be an opening that communicates with the first channel tube 571. The opening 411a and the first channel tube may be configured to receive a treatment manipulator or hardness changing device. The first opening 411a may be a circular opening in a front view when seen from the distal end. A distal end of a treatment manipulator inserted into the first channel tube 171 may protrude from, and retract into, the first opening 411a.

In some variations, the first opening 411a may comprise one or more notch portions 411n. Notch portions 411n may be formed opposite one another along a transverse direction (e.g., perpendicular, LR direction) relative to a longitudinal direction of the insertion manipulator. A notch portion 411n, which may extend radially outward from the first opening 411a on opposite sides of the first opening 411a, may be configured to receive portions of treatment manipulators 340. For example, as illustrated in FIG. 3, arms of the treatment manipulators 341 protruding from the first opening 111a toward the distal end side A1 may be positioned within the notch portions 311n. In some variations, the notch portions 311n, 411n may extend radially outward to an outer surface of the distal end portion of the treatment manipulator. In other words, a notches 311n, 411, may extend from an inner circumferential surface of the first opening 311a in a direction perpendicular to the longitudinal direction, but may not extend through an outer surface of the treatment manipulator. The size of the cross-sectional area of the opening 311a, 411a at the distal end may be larger than the cross-sectional area of the first channel (e.g., first channel tube) at a proximal or central portion of the insertion manipulator because the opening 311a, 411a has the notch 311n, 411n. In this manner, the size of the cross-sectional area of the channel (e.g., first channel) of the insertion manipulator may be different between the distal end and the proximal end of the insertion manipulator. A dimension of the notch 311n, 411n, transverse to the direction the notch extends radially outward from the first opening 411a, may be at least that of a diameter of an arm 341 of a treatment manipulator 340 that is in the first channel of insertion manipulator.

Referring to FIGS. 4 and 5, the second opening 411b may be an opening that communicates with the second channel tube 572. The second opening 411b may be a circular opening in a front view when seen from the distal end. In some variations, a distal end of the treatment manipulator inserted into the second channel tube 172 may protrude from, and retract into, the second opening 411b. Devices inserted into the second channel tube 572 may include, but are not limited to, a treatment manipulator and various manual medical instruments.

An inner diameter D1 of a part of the first opening 411a other than the notch portions 41 In may be larger than an inner diameter D2 of the second opening 411b. Specifically, the diameter D1 of the part other than the notch portions 41 In in the first opening 111a may be about three to about five times the inner diameter D2 of the second opening 411b.

The water supply nozzle 411d may be an opening that communicates with the water supply tube 575. A liquid in a tank installed in the vicinity of the control device 170 may be delivered from the water supply nozzle 411d via the water supply tube 575.

The air supply nozzle 411e may be an opening that communicates with the air supply tube 576. A gas in a tank installed in the vicinity of the control device 170 may be delivered from the air supply nozzle 41 le via the air supply tube 576.

The suction nozzle 411f may be an opening that communicates with the suction tube 577. A tank installed in the vicinity of the control device 170 may suction a gas or a liquid from the suction nozzle 411f via the suction tube 577.

As illustrated in FIG. 5, the first channel tube 571 may be a tube including a first treatment tool lumen 571r. In some variations, treatment tool lumen 571r may comprise the largest diameter of all lumens of the treatment manipulator and may have a diameter of about 50% or more the outer diameter of the treatment manipulator. For example, the inner diameter DI of the first treatment tool lumen 571r may be equal to or greater than half the outer diameter of the outer sheath 518.

The second channel tube 572 may be a tube including a second treatment tool lumen 572r. The inner diameter D1 of the first treatment tool lumen 571r may be larger than the inner diameter D2 of the second treatment tool lumen 572r. Specifically, the inner diameter D1 of the first treatment tool lumen 571r may be about three to about five times the inner diameter D2 of the second treatment tool lumen 572r. Each of the first and second treatment lumens 571r, 572r may be configured to receive a corresponding treatment manipulator and/or surgical tool. For example, FIG. 3 depicts treatment manipulators 340 inserted into the first treatment tool lumen 571r.

In some variations, the first channel tube 571 may comprise a proximal end channel tube disposed in the flexible portion 111.9 and a distal end channel tube disposed in the bending portion 111.2. The proximal end channel tube and the distal end channel tube may be connected to form the first treatment tool lumen 171r.

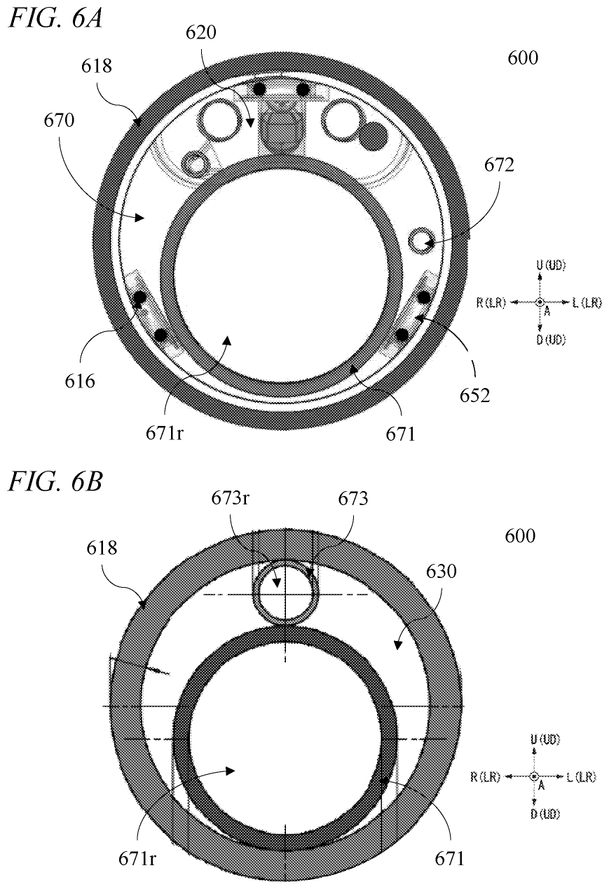

FIG. 6A depicts a cross-sectional view of a distal portion of another exemplary insertion manipulator 600. FIG. 6B depicts an illustrative variation of a simplified cross-sectional view of the insertion manipulator 600 of FIG. 6A. The insertion manipulator 600 may comprise an outer sheath 618 forming an internal path 630. In some variations, a built-in element 670 and/or one or more channels may separate the internal path 630 into two or more lumens. Any one of the built-in elements 670 or channels may be formed integrally as part of the insertion manipulator 600. The insertion manipulator 600 may comprise a first channel tube 671 (e.g., a first channel tube tube), a second channel tube 672, and a scope channel tube 673. FIG. 6A further depicts a scope 620 positioned within the scope channel tube 673.

In some variations, the insertion manipulator 600 may comprise one or more cables 616 and, optionally, one or more pulleys 652 to move or bend the insertion manipulator 600 as described in more detail herein. As shown in FIG. 6A, the insertion manipulator 600 may comprise one or more cables 616 coupled to respective pulleys 652 positioned within the internal path 630. The cables may be positioned away from a central longitudinal axis of the insertion manipulator 600 to improve the force that a cable 616 may exert on the insertion manipulator to bend the insertion manipulator. For instance, the cables 616 and/or pulleys 652 may be coupled to the outer sheath 618 as described in more detail herein. Such a configuration may enhance bending efficiency while preserving internal path space.

In some variations, the insertion manipulator 600 may comprise a scope channel tube comprising a scope lumen 673r. The scope lumen 673r may be configured to partially or fully receive a scope of the endoscope system therein. For example, the scope lumen 673r may be configured to receive an imaging cable or a light guide of the scope. Generally, the scope channel tube 673 comprising the scope lumen 673r may be positioned within the internal path 630 of the insertion manipulator 600. In some variations, the scope channel tube 673 may be integrally formed as part of the built-in element 670. The scope channel tube 673 and scope lumen 673r may be positioned to allow an imaging unit of the scope to observe the treatment manipulator 400 and/or a surgical tool coupled thereto.

The size and shape of the scope channel tube 673 and/or scope lumen 673r may vary depending on the scope used. The scope channel tube 673 and scope lumen 673r may generally be sized and shaped to accommodate the scope while minimizing the space occupied within the internal path 630 of the insertion manipulator 600. For example, the scope channel tube 673 and the lumen 673r therethrough may comprise a circular or elliptical cross-sectional shape. In some variations, the scope channel tube 673 and scope lumen 673r may comprise an elliptical or otherwise non-circular cross-section having a greater transverse dimension in a first direction (e.g., UD direction as shown in FIG. 4) and a lesser transverse dimension in a second direction (e.g., LR direction as shown in FIG. 4). Each of the transverse directions may be perpendicular to a longitudinal axis of the insertion manipulator and may be perpendicular to each other. Such cross-sectional shapes may further optimize use of the limited space within the internal path of the insertion manipulator 600. In some variations, the greater transverse dimension of the scope lumen may be greater than the diameter of the treatment lumen. As one example, the scope channel tube 673 may have an outer diameter of about 2 mm to about 5 mm, including all subranges and values therebetween, such as about 3.4 mm. The scope lumen 673r may have an inner diameter of about 1 mm to about 4 mm, including all subranges and values there between, such as about 2.8 mm.

The insertion manipulator 600 may further include a first channel tube 671 comprising a treatment lumen 671r extending therethrough. The treatment lumen 671r may be configured to receive a treatment manipulator to deliver the treatment manipulator to a target site within the body of a patient. The first channel tube 671 may also be configured to receive other devices or materials in addition to a treatment manipulator. In some variations, the first channel tube and treatment lumen may be the largest channels and lumens of the insertion manipulator, respectively. Different cross-sectional shapes may be used for the treatment lumen, including circular, elliptical, or square cross-sectional shapes, depending on the type of tool or device intended for passage through the channel. A circular cross-sectional shape may be advantageous to reduce being stiffness of the insertion manipulator. The first channel tube 671 may, for example, have an outer diameter of about 8 mm to about 15 mm, including all subranges and values therebetween, such as about 11.6 mm. An inner diameter the treatment lumen may be about 6 mm to about 12 mm, including all subranges and values therebetween, such as about 10 mm to accommodate a treatment manipulator or other surgical device.

The first channel tube 671 and treatment lumen 671r comprise therein may be positioned within the internal path 630 of the insertion manipulator relative to the scope channel tube 673 and scope lumen 673r comprised therein to optimize use of the limited space within the insertion manipulator 600. Generally, the inner diameter of the treatment lumen 671r may be greater than or equal to the greater (e.g., maximum) transverse dimension of the scope lumen.

The scope lumen 673r and the treatment lumen 671r may be located on a same central axis perpendicular (e.g., along the UD direction) to a longitudinal axis (e.g., A) of the insertion manipulator 600. The central axis may be an inner diameter of the insertion manipulator. In some variations, the inner diameter of the treatment lumen may be less than the outer diameter of the outer sheath 618 less a transverse dimension of the scope lumen. Put another way, the inner diameter of the treatment lumen may be given by the equation: ID_{treatment lumen}≤OD_{outer sheath}−ID_{scope lumen}.

The first channel tube 671 may be formed independently of the outer sheath 618 and may remain unsecured to the outer sheath to allow relative flexibility and motion. In other variations, the first channel tube 671 may be secured to the outer sheath 618 or a built-in element 670. The first channel tube 671 may have greater stiffness than the outer sheath 618, which may assist with advancement and device stability. To reduce kinking and maintain lumen patency, the wall of the first channel tube 671 may incorporate a stiffener, such as, for example, one or more of a coil or a braid. In some variations comprising a braid, the braid may comprise nitinol wire. The outer sheath may comprise a braid or a coil held under a compressive load to further support the lumen shape.

In some variations, a separation may be maintained between the first channel tube 671 and the scope channel tube 673 so that the scope 620 can observe the treatment manipulator or tools coupled thereto without obstruction. For example, a portion of a built-in element 670 may separate the scope channel tube 673 and first channel tube 671.

The insertion manipulator 600 may include an outer sheath 618 configured to contain the first channel tube 671, the scope channel tube 673, and any built-in elements or additional channels and lumens described herein. The outer sheath 618 may form the exterior of the insertion section and may be the portion that contacts a patient when the insertion manipulator is introduced into the body. In certain variations, the outer sheath 618 may incorporate one or more stiffeners, such as a spring, which are described in more detail herein.

Dimensions of the outer sheath 618 and the insertion section may vary depending on the intended clinical application. For example, in a system configured for use in a colonoscopy, the outer diameter of the outer sheath 618 of the insertion section may range from about 20 mm to about 17 mm, including all subranges and values therebetween, such as about 18.2 mm or about 20 mm. The wall of the outer sheath 618 may have a thickness of about 1 mm to about 2.5 mm, including all subranges and values therebetween, such as about 1.6 mm. In variations in which the insertion section does not include a scope channel tube 673 or scope lumen 673r, the outer diameter of the insertion section may be reduced, for example to about 15 mm to about 19 mm, including all subranges and values therebetween, such as about 17.2 mm or about 17.4 mm. A reduced diameter may also correspond to a lower bending stiffness of the insertion manipulator, which may advantageously allow the insertion manipulator to be more easily steered.

### Bending Portion

As shown in FIG. 3, the insertion manipulator may comprise a bending portion 312. The bending portion may be a central portion of the insertion section, positioned between the distal end portion 311 and a flexible proximal portion (e.g., 111.9 of FIG. 1). The bending portion may be configured to manipulate the distal end (e.g., distal end portion 311) of the insertion manipulator to reach a target site within a patient. In some variations, the bending portion 312 may comprise a length of about 80 mm to about 120 mm, including all subranges and values therein, such as about 80 mm, about 100 mm, or about 120 mm. Furthermore, the bending portion 312 may be configured to bend about 0 degrees to about 90 degrees, about 0 degrees to about 180 degrees, about 30 degrees to about 180 degrees, up to about 90 degrees, or up to about 180 degrees, or up to about 200 degrees, including all subranges and values in any of the foregoing.

The bending portion 312 may include a plurality of joint rings (i.e., bending pieces) 315, a distal end portion 316 connected to a distal end of the plurality of joint rings 315, and an outer sheath (not shown). The plurality of joint rings 315 may be connected in the longitudinal direction within the outer sheath. A joint ring 315 at the distal end of the bending portion 312 may be connected to the distal end portion 311.

A joint ring may be a short cylindrical member. In some variations, the joint ring may be formed of metal. Multiple joint rings may be connected such that internal spaces of neighboring joint rings form a continuous space. Each joint ring may include a first joint ring 315a on the distal end side and a second joint ring 315b on the proximal side. The first joint ring 315a and the second joint ring 315b may be connected by a first rotary pin so as to be rotatable in a vertical direction (e.g., UD direction as shown in FIG. 4) perpendicular to the longitudinal direction.

In some variations, one or more wire-guides may be formed on or coupled (e.g., welded) to one or more of the joint rings. For instance, an upper wire-guide and a lower wire-guide may be formed on an inner circumferential surface of the second joint ring 315b. The upper wire-guide and the lower wire-guide may be disposed in opposite lateral directions with a central longitudinal axis of the insertion manipulator interposed therebetween. Additionally or alternatively, a left wire-guide and/or a right wire-guide may be formed on or coupled to an inner circumferential surface of a first joint ring. The left wire-guide and the right wire-guide may be disposed in opposite lateral directions with a central longitudinal axis of the insertion manipulator interposed therebetween. The opposing directions of the left wire-guide and right wire-guide may be perpendicular to the opposing lateral directions of the upper and lower wire-guides. In some variations, a wire-guide may comprise one or more lumens into which a bending wire (i.e., cable) 316 is inserted. The bending wire may extend parallel to the central longitudinal axis of the insertion manipulator.

The cable 316 (i.e., bending wire) may be a wire configured to bend the bending portion 312. The cable 316 may extend to the attachment portion 115 through an internal path of the insertion section 310. An insertion manipulator may include a plurality of cables, such as, for example, an upper bending wire, a lower bending wire, a left bending wire, and/or a right bending wire. Each cable may be guided by one or more wire-guides configured to maintain the cable path within the internal passage. In some variations, a wire-guide may include a ferrule or other structure configured to reduce chafing of the cable. Additionally or alternatively, a wire-guide may comprise a metal (e.g., stainless steel), ceramic (e.g., toroidal ceramic bushings), and/or a plastic (e.g., PLA). In some variations it may be advantageous to include a wire-guide that comprises a hard material, such as a metal and/or a ceramic, to reduce friction between the guide the cable.

An upper bending wire and a lower bending wire may bend the bending portion 312 in a UD direction, as shown in FIG. 4. The upper bending wire may pass through an upper wire-guide, and the lower bending wire may pass through a lower wire-guide. Similarly, a left bending wire and a right bending wire may bend the bending portion 312 in an LR direction, also shown in FIG. 4. The left bending wire may be inserted into a left wire-guide, and the right bending wire may be inserted into a right wire-guide.

Some variations of the insertion manipulator may include two, three, four, or more than four cables. Each cable may be configured to bend the bending portion 312 toward a side of the bending portion where the cable is tensioned. A distal end of a cable 316 may be fixed to a distal end portion 318 of the bending portion 312. The bending portion 312 may bend in a desired direction by pulling or relaxing the bending wires 316 corresponding to that direction. This arrangement may enable controlled articulation of the distal end during endoscopic procedures.

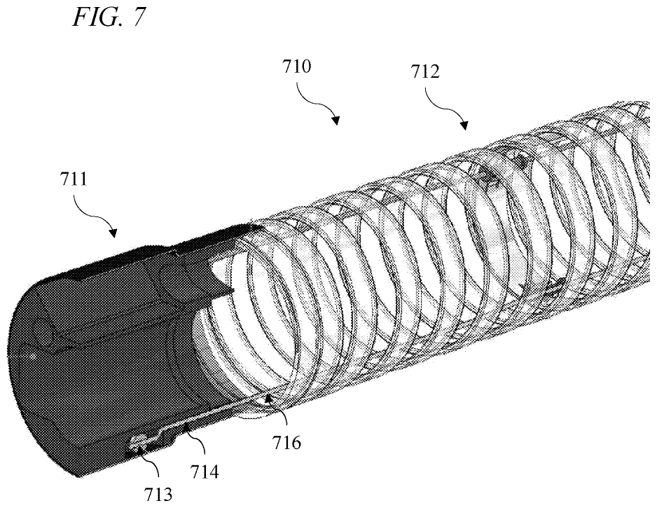

Additionally or alternatively, in some variations, one or more cables (i.e., bending wires) may be directly coupled to a distal end portion of the insertion section. As shown in FIG. 7, an insertion section 710 may comprise a distal end portion 711, a bending portion 712, and a bending wire 716 coupled to the distal end portion 711. The bending portion 712 may be bendable to move the distal end portion 711 in a desired direction by pulling or relaxing a cable 716 corresponding to that direction. As shown in FIG. 7, the cable 716 may be inserted into the distal end portion 711 to couple the cable to the distal end portion 711. To couple the cable 716 to the distal end portion 711, the cable 716 may be at least partially positioned within an opening (e.g., slot) 714 formed in the distal end portion 711. The cable may be retained by a stop 713 (e.g., crimp, molded ball) configured to secure the cable 716 within the opening 714. For example, the opening 714 may include a proximal end having a first diameter and a distal cavity having a second diameter larger than the first diameter. To couple the cable 716 to the distal end portion 711 via the opening 714, the proximal end of the opening may have a maximum transverse dimension greater than that of the cable but less than that of the stop 713, allowing the cable 716 to pass through the proximal opening while passage of the stop is prevent and the stop remains in the distal cavity of the opening.

In some variations, the bending portion may be configured to bias movement (e.g., deflection or bending) toward a distal region of the bending portion. Improved bending in the distal region can be desirable to provide better control of the distal end portion of the insertion section and/or a treatment manipulator positioned within or coupled to the insertion section. Conventional endoscopes often have difficulty creating tight bends in their distal portions or even achieving uniform bending along their length. These deficiencies may arise from factors such as cable friction or the inherent stiffness of the endoscope.

An insertion manipulator configured to bias movement of the bending portion toward the distal end, as described herein, may therefore enable increased control (e.g., tighter bends in the distal region or more uniform bending along the entire insertion section). To accomplish this, the bending portion may comprise a stiffness that varies along its length. For example, a proximal portion of the bending portion may have a greater bending stiffness than a distal portion of the bending portion. This configuration may be configured to bias bending toward the less stiff distal region because an applied cable force may have a greater effect where stiffness is lower.

In some variations, the insertion section may comprise a pulley to bias bending of the bending portion in a distal portion of the bending portion. For example, with respect to the insertion section 310 of FIG. 3, the pulley may be coupled to a proximal end of the distal end portion 311 (e.g., a distal tip) or a distal end portion 318 of the bending portion 312 (e.g., central portion). To apply a bending force, a cable may be anchored in a distal portion of the bending portion 312 and coupled to the pulley. The cable may further run from the pulley to a proximal portion of the insertion manipulator (e.g., 119, 150). The pulley may increase the force applied by the cable to the distal portion of the bending portion when tension is applied to a proximal end of the cable. This configuration may thus improve the ability of the distal portion of the bending portion to articulate (bend) in response to tension from the cable.

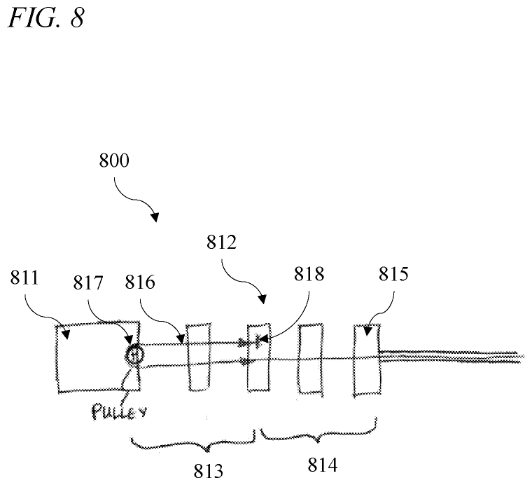

FIG. 8 depicts a portion of an insertion section 800 of an insertion manipulator including a pulley 817. As shown in FIG. 8, the insertion manipulator may comprise a distal end portion 811 (e.g., distal tip), a pulley 817 coupled to the distal end portion, a bending portion 812 divided into a distal portion 813 and a proximal portion 814 (e.g., central portion adjacent to the distal portion 813), and a cable 816. As shown in FIG. 8, the bending portion 812 may comprise one or more joint rings 815. The joint rings 815 may be arranged to permit bending about desired directions (e.g., a direction lateral to a longitudinal axis of the insertion manipulator, UD direction, LR direction) when tension is applied to the cable 816. The cable 816 may be coupled to the joint rings 815 by one or more wire-guides as previously described herein. Additionally, the cable 816 may be radially offset from a longitudinal central axis of the insertion manipulator 800 to improve the bend force applied to the insertion manipulator by the cable 816.

In some variations, the force applied by the cable 816 to the distal portion 813 may be greater than (e.g., double) the force applied to an adjacent a proximal portion 814 of the bending portion. For instance, because two portions of the cable 816 may reside in the distal portion 813 of the bending portion 812 compared to only the single portion in the proximal portion 814, the distal portion 813 may be subject to double the force of the proximal portion 814. In some variations, the cable 816 may include a first portion that is coupled to and runs from an anchor 818 in the distal portion 813 to the pulley. The cable may further comprise a second and a third portion (e.g., a second portion in the distal portion 813 and a third portion in the proximal portion 814) that run (e.g., consecutively) from the pulley 817 to a proximal end of the insertion section. The anchor 818 and/or pulley 817 may increase the force applied by the cable to the distal portion when tension is applied to the proximal end of the cable. In certain configurations, the applied force may bend the insertion section or endoscope by about 180 degrees or more.

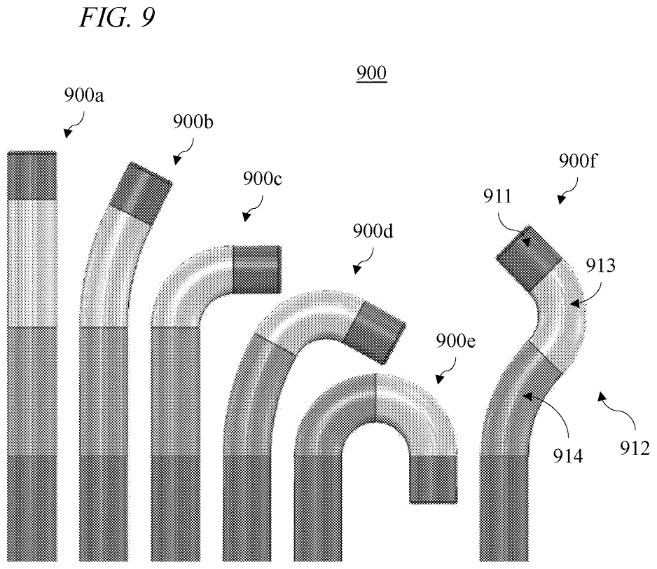

In some variations, the bending portion may comprise a first bending portion and a second bending portion, where the first and second bending portions may be configured to move independently of one another. Independent bending of a first and second portions may improve maneuverability of the endoscope within a patient. For example, FIG. 9 depicts an insertion section 900 of an insertion manipulator including a distal end portion 911 and a bending portion 912 having a first bending segment 913 and a second bending segment 914, each operable independently of the other. Each independent bending segment 913, 914 may comprise a respective plurality of cables (e.g., three cables per portion) to move (e.g., bend) that segment. The bending segments being configured to move independent of one another may allow the first and second bending segments to bend to different angles from one another.

For example, configurations 900a, 900b, 900c, and 900d of FIG. 9 show the first and second bending segments at different degrees of bend. In configuration 900a, the first bending segment 913 and the second bending segment 914 are each in a straight configuration and may be substantially collinear with a longitudinal axis of the insertion manipulator. In this configuration 900a, neither segment 913 nor segment 914 comprises a curvature, and the distal end portion 911 is held in alignment with the insertion section 900. The angular deflection of each of segments 913, 914 relative to the longitudinal axis may be about 0 and a combined deflection of the bending portion 912 may likewise be about 0°.

In configuration 900b, the second bending segment 914 may be straight and axially aligned with the insertion manipulator, while the distal, first bending segment 913 is bent to define a curvature. In this configuration, the first segment 913 may be angled by about 10° to about 45° relative to the longitudinal axis of the insertion manipulator, including all subranges and values therebetween, such as about 15°, about 30°, or about 40°, while the second segment 914 remains at about 0°. A radius of curvature may be defined by the first segment 913, whereas the second segment 914 may comprise no curvature. Accordingly, the combined deflection of the bending portion 912 may correspond to the curvature of the first segment 913 (e.g., about 10° to about 45°).

In configuration 900c, the second bending segment 914 may again be straight and collinear with the longitudinal axis of the insertion manipulator, while the first bending segment 913 alone is bent to define a curvature. However, the angle of the first segment 913 in configuration 900c is greater than that of configuration 900b, for example about 60° to about 120°, including all subranges and values therebetween, such as about 90°, relative to the longitudinal axis. In configuration 900c, only the first segment 913 defines a curvature, and the combined deflection of the bending portion 912 may be substantially equal to the deflection of the first segment 913 (e.g., about 60° to about 120°, about 90°).

In configuration 900d, both the first bending segment 913 and the second bending segment 914 may be bent relative to the longitudinal axis of the insertion manipulator. The first bending segment 913 may be angled by an amount within the same range as described for configuration 900c (e.g., about 60° to about 120°, such as about 90°), and the second segment 914 may be angled by about 10° to about 45°, including all subranges and values therebetween, such as about 15°, about 25°, or about 35°, relative to the longitudinal axis. In some variations, the first segment 913 may be bent to a greater angle than the second segment 914 and may define a smaller radius of curvature than the second segment 914 (e.g., tighter curvature distally with a comparatively larger, more gradual curvature proximally). The combined deflection of the bending portion 912 in configuration 900d may be about 90° to about 135°, such as about 100°, about 120°, or about 130°, depending on the selected angles of the first and second segments

In configuration 900e, both the first bending segment 913 and the second bending segment 914 are bent and each defines a respective curvature. In some variations, each of the segments 913 and 914 may be angled by about 60° to about 90° relative to the longitudinal axis of the insertion manipulator (e.g., the first segment 913 at about 60° about 90° and the second segment 914 at about 60° to about 90°). The radii of curvature of the segments 913 and 914 may be similar to one another, or, in some variations, the first bending segment 913 may define a smaller radius of curvature (tighter bend) than the second bending segment 914. In additional variations, each bending segment 913, 914 may be actuated to the same bend angle to achieve an approximately uniform bend along the bending portion 912 (e.g., a substantially constant radius of curvature along the length of bending portion 912). For instance, when each bending segment 913, 914 is moved to bend maximally in the same direction, the bending portion 912 of the insertion section may bend 90 degrees or more, such as 180 degrees or more, as shown in configuration 900e of FIG. 9. The combined deflection of the bending portion 912 in configuration 900e may therefore be about 120° to about 180°, including all subranges and values therebetween.

In configuration 900f, the independent bending segments 913 and 914 may be actuated to bend in different directions. For example, the first bending segment 913 may bend in a first direction while the second bending segment 914 may bend in a second, different (e.g., opposite) direction, thereby forming a “nod” configuration as depicted in FIG. 9. In some implementations, each segment 913, 914 may be bent to any of the angular ranges described herein (e.g., about 10° to about 90°, about 10° to about 45°, about 60° to about 120°, or about 60° to about 90°), and each may define its own radius of curvature, which may be similar or different from that of the other segment. This directional opposition between the first and second bending segments may allow the distal end portion 911 to articulate in a manner advantageous for accessing anatomically difficult-to-reach regions, steering around obstructions, or providing enhanced navigational control within tortuous pathways.

In each of configurations 900a through 900f, the first and second bending segments 913, 914 may be actuated independently (e.g., by respective pluralities of control cables). It should be appreciated that, in some variations, a bending portion may comprise additional independent bending portions to allow for further control over steering of the insertion manipulator. For example, an insertion manipulator may comprise two, three, four, or more than four different independent bending portions configured to move in different directions.

### Sheath

As referenced previously herein, the insertion manipulator may further compromise an outer sheath. Generally, the outer sheath may be configured to protect other components of the insertion manipulator and may form the outermost surface of the insertion manipulator (e.g., the surface that contacts a patient). However, in some variations, the outer sheath may be further configured to provide additional structural support to the insertion manipulator. For example, when a channel tube of insertion manipulator (e.g., first channel tube) does not have sufficient kink-resistance, torque transmissivity, and/or pushability, additional structural support may be desirable. The outer sheath may include a multi-layered structure including a support (e.g., spring) and/or braided tube, which may supply torsional rigidity so the insertion manipulator may be rotated while positioned within a patient. In some variations, the support may have a low bending stiffness to maintain flexibility of the insertion section.

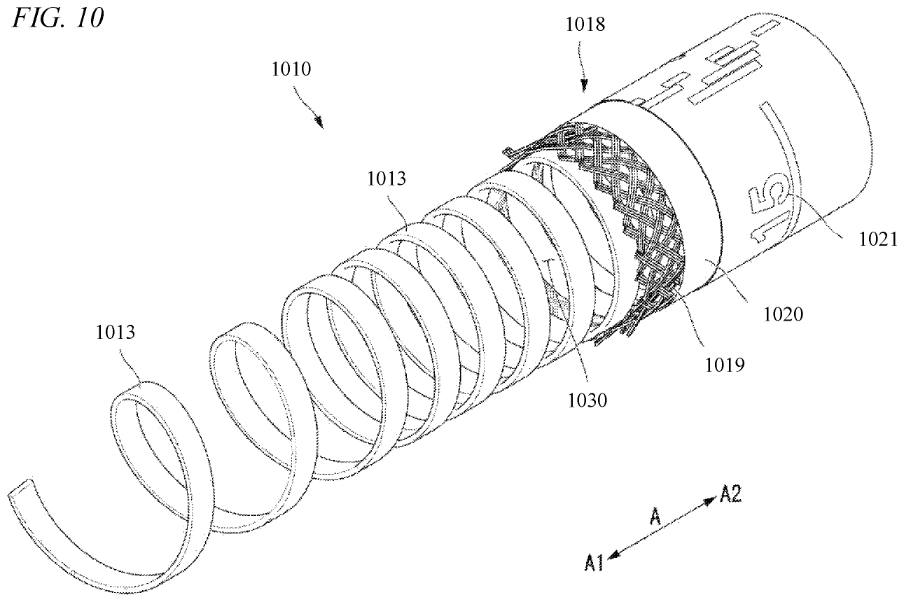

FIG. 10 depicts an exemplary outer sheath 1018 of an insertion manipulator. For example, as shown in FIG. 10, the insertion section 1010 may include a support comprising a spring 1013. The spring 1013 may be formed by winding a linear metal member in a spiral shape. The spring 1013 may be a flat-wire spring or a round-wire spring. Using a flat-wire spring instead of a round-wire spring may help reduce the diameter of the insertion section 1010. Conversely, using a round-wire spring may make the insertion section 1010 easier to bend. For clarity, cables and the built-in element are omitted in FIG. 10.

In some variations, the spring 1013 may extend from the distal end to the proximal end of the insertion section 1010. As a result, the insertion section 1010 may exhibit good torque transmission along its length. In some variations, the spring 1013 may be arranged in an internal path 1030 of the insertion section 1010 in a preloaded state with a compressive force of, for example, about 20 N to about 25 N. Because the spring 1013 may be preloaded, it may have improved kink-resistance and may resist buckling during use.

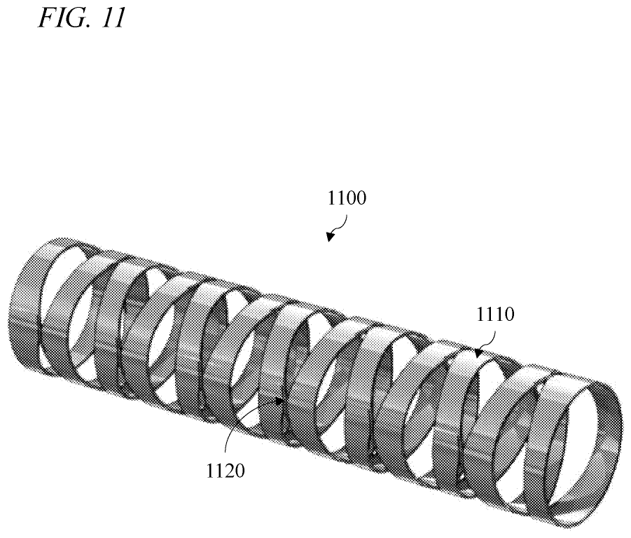

In some variations, a support of the sheath may comprise a spine rather than a spring. The spine may include two or more rings or two or more vertebrae moveably coupled to one another. For example, the spine may comprise two or more rings or vertebrae coupled by pinned joints allowed relative movement between the rings or vertebrae. Additionally or alternatively, the spine may include two or more rings or two or more vertebrae coupled by hinges (e.g., living hinges, flexures). FIG. 11 depicts an exemplary spine 1100 of an outer sheath. The spine 1100 may comprise a plurality of vertebrae 1110 moveably coupled to one another (e.g., by living hinges 1120). As shown in FIG. 11, the vertebrae 1110 and hinges 1120 of the spine may be integrally formed from a single tube. For example, the spine 1100 may be laser-cut from a Nitinol tube. In contrast to utilizing a spring for support, a spine 1100 may have a fixed center-line bending length. However, the length along an outer surface of an outer sheath comprising a spine 1100 may increase or decrease based on the curvature of the outer sheath and spine 1100. This may cause material on the outer surface of the outer sheath to experience strain of up to about 40%. The sheath may comprise materials such as elastomers configured to accommodate such strains without permanent deformation.

In some variations, a spine may have a lower bending stiffness compared to that of a spring. In such variations, the insertion manipulator may be configured to actuate (e.g., bend) in response to significantly lower cable forces, which in turn may reduce friction effects. Furthermore, in variations of an insertion manipulator comprising a spine, the outer sheath may not include a braid, and the spine may provide the necessary torsional stiffness.

Turning back to FIG. 10, an outer sheath 1018 may be positioned on the outside of the spring 1013. The outer sheath 1018 may include a braided tube 1019 and a coating 1020 on an inner and/or outer side of the braided tube 1019. In some variations, a distal portion of the braided tube 1018 may be coupled to a rigid portion of the distal end portion of the insertion section. The coating 1020 positioned on the inner side may be referred to as an inner coating, and the coating 1020 positioned on the outer side may be referred to as an outer coating. Either the inner coating, the outer coating, or both may be provided depending on the desired properties.

The braided tube 1019 may be formed by weaving metal wires, resin wires, or similar fibers into a braid. The braided tube 1019 may be composed of, for example, aramid fibers or Ultra High Molecular Weight Polyethylene (UHMW-PE).

The coating 1020 may be positioned on the inner and/or outer side of the braided tube 1019 to encapsulate and seal the braided structure. The coating 1020 may be joined to the braided tube 1019 by coating, co-extrusion, over-casting, or over-molding. The coating 1020 may be formed of a low-friction, low-durometer resin.

In the outer sheath 1018, the braided tube 1019 may be encapsulated by the coating 1020, which may allow the braided structure to maintain rigidity without increasing the diameter of the insertion section. Because the coating 1020 may have low friction and a low durometer, the outer sheath 1018 may achieve a balance of durability, elasticity, and ease of bending.

The outer surface of the outer sheath 1018 may include a marking 118m that can be observed under X-ray fluoroscopy. By confirming the marking 1021, a surgeon S may readily determine the insertion position and insertion depth of the insertion section 1010 during a procedure.

An outer sheath 1018 as described herein may form part of any one of the distal end portion 111.1, the bending portion 111.2, or the flexible portion 111.9 of the insertion section.

### Treatment Manipulator

A treatment manipulator may be used to engage, position, or otherwise act upon tissue at a target site within a patient. The treatment manipulator may be delivered to the treatment site via an insertion manipulator as described herein. For instance, the treatment manipulator may be positioned (e.g., slidably positioned) at least partially within a treatment lumen of the insertion manipulator. The distal portion of the treatment manipulator may extend beyond the distal end of the insertion manipulator to access the target site. Working in combination, the insertion manipulator may be configured to guide the system within the body, while the treatment manipulator may provide refined, localized movement for performing therapeutic or diagnostic operations (e.g., to treat a lesion).

In some variations, the treatment manipulator may include one or more bendable arms configured to articulate in one or more directions. The arms may include one or more actuators configured to apply controlled forces to bend, rotate, and/or position the arms relative to the surrounding anatomy and/or endoscope system. For example, in some variations, the arms may be actuated by one or more artificial muscles. Through such articulation, the treatment manipulator may provide for precise positioning of one or more treatment tools at the target site.

A treatment manipulator 340 coupled to (e.g., received within) an insertion manipulator is shown in FIG. 3. Generally, the treatment manipulator 400 may comprise a bendable treatment tool arm 341 that may comprise, or may be configured to receive, a treatment tool 342 (e.g., a forceps, a high-frequency knife, a local injection needle, a basket, etc.). For example, the treatment tool arm 341 may comprise an elongate member comprising a lumen therethrough. Similar to the bending portion of the insertion manipulator, the treatment tool arm 341 may comprise a plurality of joint rings (also referred to as bending pieces) and may be driven with an actuator, such as, for example, a wire, an artificial muscle, or the like, such that it is be bendable in one or more directions (e.g., a vertical direction and/or a lateral direction). The treatment tool 342 may be positioned within the lumen of the treatment tool arm 341. A treatment tool 342 or portion thereof may be passively bendable (e.g., flexible) and may not have an active bending function.

The treatment manipulator 340 may be sized to fit within a treatment lumen on the insertion section 310. For instance, in a delivery configuration (not shown in FIG. 3), the treatment manipulator 340 may comprise an outer dimension that is less than the inner diameter of the treatment lumen to allow the treatment manipulator to be inserted therein. As described previously herein, a treatment lumen of the insertion section may comprise a large inner diameter (e.g., about 10 mm). In some variations, the treatment tool lumen may be configured to receive a treatment manipulator comprising two or more arms.

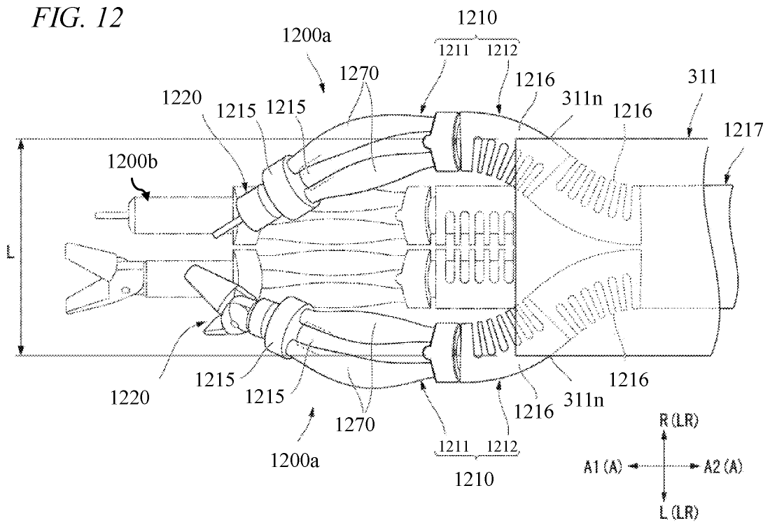

FIG. 12 shows a top view of an exemplary a treatment manipulator 1200. FIG. 12 shows the treatment manipulator in a delivery configuration 1200b and an operating configuration 1200a. In some variations, the treatment manipulator 1200 is driven by a drive unit (treatment manipulator actuator) or the like. The treatment manipulator 1200 may comprise two arms 1210 that are arranged on a distal end surface of a manipulator flexible portion 1217. In some variations, an arm may be coupled at a proximal end to a rigid base (not shown) which may be coupled to the distal end of the manipulator flexible portion 1217. Herein a single arm of a treatment manipulator may be discussed for purposes of clarity and brevity. Likewise, one or more arms may be described as if they are identical to one another for the explanation of particular features or configurations. However, it will be understood by a person of ordinary skill in the art that the treatment manipulator may include multiple arms that need not be identical or similar, and that any individual arm may incorporate different combinations or subsets of the features described herein with respect to a single arm or with respect to multiple arms. Accordingly, the descriptions herein should not be construed as limiting the treatment manipulator to arms having similar construction or behavior unless expressly stated otherwise

The treatment manipulator 1200 may comprise or be configured to receive one or more treatment tools 1220, such as a forceps, a high-frequency knife, a local injection needle, and/or a basket. Moreover, the treatment manipulator may further comprise a treatment tool arm 1210 and actuator which may comprise an artificial muscle 1270 and/or a cable.

As mentioned above, the treatment tool arm 1210 may comprise an elongate member. In some variations, the treatment tool arm 1210 may include a first (e.g., distal) bending portion 1211 and a second (e.g., proximal) bending portion 1212. As shown in the operating configuration of the treatment manipulator 1200 illustrated in FIG. 12, the first bending portion 1211 may be positioned distal to a distal end surface of the distal end portion 311 when the treatment manipulator 1200, or a tool received therein, is in use during a procedure. A portion of the second bending portion 1212 may protrude distally from the distal end portion 311.

The first bending portion 1211 of each arm may comprise a plurality of joint rings (also referred to herein as wrist joints) 1215 similarly to the bending portion of the insertion manipulator. The first bending portion may be actuated by one or more a first actuators, such as one or more first artificial muscles 1270, such that it is bendable in one or more directions (e.g., a vertical direction, a lateral direction). Additionally or alternatively, the second bending portion may be actuated by one or more second actuators, such as one or more artificial muscles, as described herein in more detail. Put another way, in some variations, a first plurality of artificial muscles may be configured to actuate only the first bending portion and second plurality of artificial muscles may be configured to actuate only the second bending portion. However, in certain other variations, the same plurality of artificial muscles may be configured to actuate the first and the second bending portions.

As illustrated in FIG. 12, the second bending portion 1212 of each arm (e.g., proximal bending portion) may include a shoulder joint 1216 configured to allow the arm 1210 to bend in a lateral direction (“LR direction”). In some variations, the shoulder joint 1216 of each may be configured to bend the second bending portion 1212 in an S-shape when viewed from a top view (i.e., in the vertical direction (UD direction)). As illustrated in FIG. 12, a distal end of the second bending portion 1212 of each treatment tool arm 1210 may be bent to separate the distal end of each second bending portion from one another in a lateral direction. The distance between the center axes of each arm 1210 at the distal ends of the second bending portions 1212 may be defined as distance L (shoulder width). The arms may be configured to separate a distance L sufficient for treatment. In particular, proximal ends of the two first bending portions 1211 may be separated to treat a target site within a patient. To achieve sufficient separation, at least a portion of each arm 1210 may be received into notch portions 31 In and spread outward in the lateral direction. Accordingly, the distance L may be larger than the outer diameter of the distal end portion 311 of the insertion manipulator.

An artificial muscle 1270 may comprise at least a part of a mechanism for bending an arm and/or treatment tool (e.g., surgical tool) coupled to the arm. The artificial muscle 1270 may be, for example, a Mckibben artificial muscle. One or more artificial muscles 1270, as shown in the exemplary treatment manipulator 1200 of FIG. 12, may be coupled to the first bending portion 1211 of one or both treatment tool arms 1210 and may be configured to bend the first bending portion 1211 of one or both respective treatment tool arms 1210. In other variations described herein, one or more artificial muscles 1270 may be coupled to the second bending portion 1212 of one or both treatment tool arms 1210. In order to bend the first and/or second bending portions 1211, 1212 in the vertical direction and/or the lateral direction, a plurality (e.g., two, three, four, or more) artificial muscles 1270 may be coupled to one or both of the treatment tool arms 1210.

### Artificial Muscles

Generally, an artificial muscle may comprise an elongate body configured to lengthen and contract and/or expand and contract laterally. A controlled lengthening and contraction of the elongate body of an artificial muscle, or of a plurality of artificial muscles, may actuate an arm of the treatment manipulator. For example, the elongate body may be configured to change length in response to a fluid (e.g., a pressurized fluid) being provided to the artificial muscle. In some variation, the elongate body may be filled with the fluid. The elongate body may comprise one or more layers of an elastic material, such as an elastomer, to allow for a change in the length and/or transverse dimension of the artificial muscle. Moreover, the elongate body may comprise a braided layer configured to control dimensional changes of the elongate body. For instance, the braided layer may restrict elongation of the elongate body or may cause the elongate body to shorten when the elongate body is pressurized with a fluid. Each of the layers may comprise a tube shape and the tubular layers may be arranged around one another (e.g., concentrically) to form a tubular elongate body.

The elongate body may comprise a lumen for receiving a fluid. To hold the fluid, particularly under pressure, the elongate body may be sealed at one or both ends. To seal the elongate body, the artificial muscle may comprise one or more of a port, an end cap, and/or a crimp. An artificial muscle port may be positioned at least partially within the lumen of the elongate body. For example, the port may comprise a body configured to be positioned into the lumen to seal the lumen. An end cap of the artificial muscle may comprise a similar structure to the port to seal an end of the elongate body lumen. In some variations, the and cap and/or port may further comprise a flange, however, in these variations, the port flange may be configured to couple the port to a fluid input pipe, while the end cap flange may not. Additionally, the port may comprise a lumen in fluid communication with a fluid input pipe and the lumen of the elongate body so that the artificial muscle may receive fluid and/or changes in fluid pressure via the port lumen. In variations in which comprise an end cap, the end cap may be solid and may not comprise a fluid lumen therethrough. In some variations, the end cap may have the same configuration (e.g., geometry, features) as the port, but without a lumen. A crimp may positioned around the elongated body and may be configured to secure and fluidically seal the elongate body to the port or end cap so that the lumen of the elongate body remains sealed.

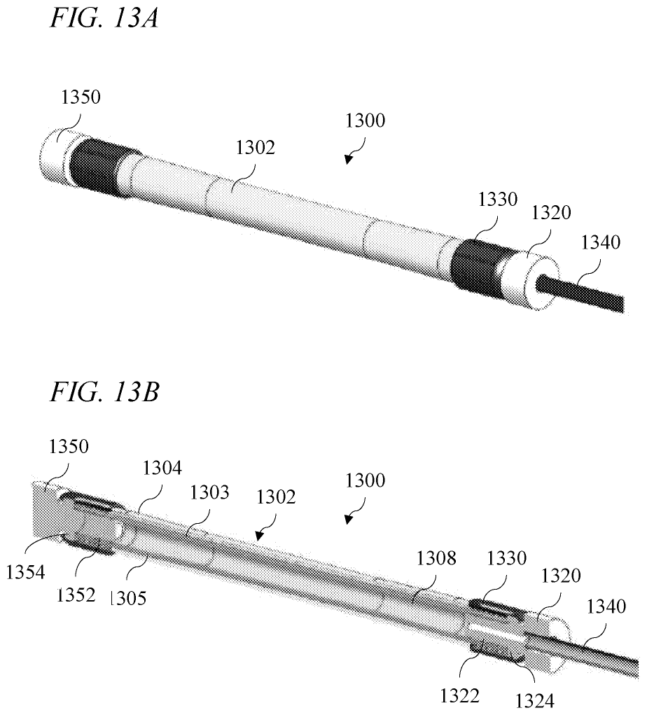

FIG. 13A depicts a perspective view of an exemplary artificial muscle 1300, and FIG. 13B depicts a perspective cross-sectional view of the artificial muscle 1300 shown in FIG. 13A. The artificial muscle 1300 may comprise a tubular elongate body 1302 that may include a first elastomer layer 1303 forming an elongate body lumen 1308, a second elastomer layer 1304 around the first layer, and a braided layer 1305 positioned between the first and second elastomer layers 1303, 1304. The first elastomer layer 1303 may function as a compliant internal bladder configured to expand or contract under fluid pressure. The second elastomer layer 1304 may provide external reinforcement and protection. The braided layer 1305 may act as a constraint structure that governs how the elongate body 1302 changes dimension under pressurization. The elastomer layers 1303, 1304 may each have a thickness of about 0.2 mm. The braided layer 1305 may comprise a liquid crystal polymer (LCP) fiber (e.g., Vectran) or aromatic polyamide fibers (i.e., Aramid), or other suitable high-strength fibers. As described previously, the elongate body lumen 1308 may be configured to receive a fluid to change a dimension of the artificial muscle 1300. In some variations, the lumen 1308 may have a diameter of about 0.5 mm. When fluid is introduced, the elongate body 1302 may shorten, and the braided layer 1305 may convert radial expansion forces into controlled axial contraction while limiting undesired longitudinal expansion.

The artificial muscle 1300 may further include a port 1320 positioned at least partially within the elongate body lumen 1308. The port 1320 may comprise an engagement feature 1322 and a locking feature 1324 configured to seal and secure the port 1320 within the elongate body 1302, respectively. A crimp 1330 may be coupled to a proximal portion of the elongate body 1302 and the port 1320. The crimp 1330 may seal the elongate body 1302 to the port 1320 through contact with the engagement feature 132 and locking feature 1324, thereby maintaining a fluid-tight interface between the port 1320 and the lumen 1308.

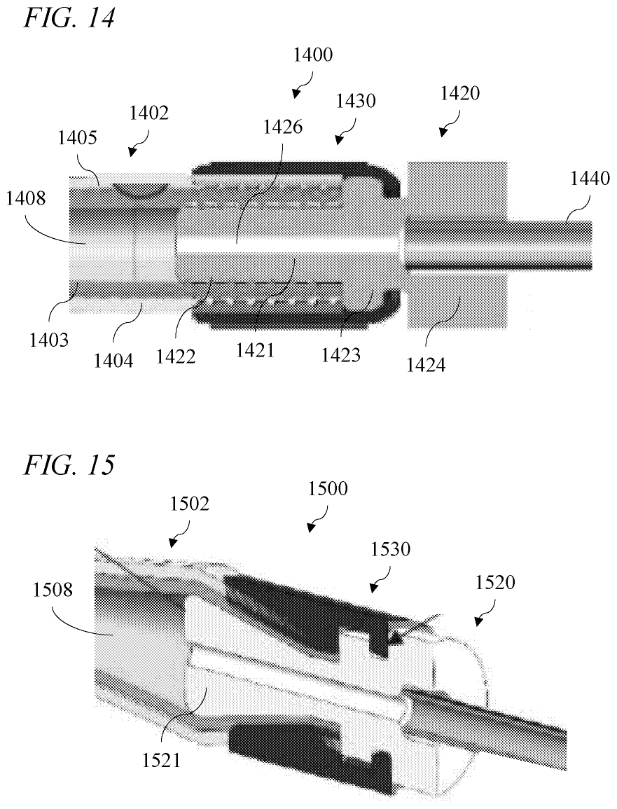

FIG. 14 illustrates a cross-sectional view of an end portion of an artificial muscle 1400 that includes an elongate body 1402 including first and second elastomer layers 1403, 1404 and a braided layer 1405. The artificial muscle 1400 may further comprise a port 1420, a crimp 1430, and a fluid input pipe 1440. The port 1420 may seal an end of the elongate body lumen 1408 and may fluidly couple the elongate body lumen 1408 and the fluid input pipe 1440. The port 1420 may comprise a rigid material, such as a metal (e.g., stainless steel). As mentioned previously, the port 1420 may comprise one or more engagement features 1422 configured to engage an inner surface of the elongate body 1402 (e.g., an inner surface of the second elastomer layer 1403) and seal the lumen 1408. Generally, a compressive force may seal the contact between the elongate body 1402 and the port 1420 and/or end cap. For instance, the elastomer layers 1403, 1404 of the elongate body may be configured to provide radial compression of the elongate body 1402 against the port 1420 when around (e.g., stretched over) the port 1420. Additionally, the crimp 1430 may be configured to radially compress the elongate body 1402 against the port to create a seal between the surfaces of elongate body 1402 and port 1420.

A port and/or an end cap may comprise an engagement feature such that the port has additional surface area compared to a featureless (e.g., smooth) port to improve a seal between the elongate body and port. The engagement feature 1422 may include one or more grooves, barbs, teeth, and/or threads (e.g., bolt threads). The engagement feature 1422 or a plurality of engagement features 1422 may be on an outer surface of the port body 1421 and/or may extend around a circumference of the port body 1421. In some variations, the crimp 1430 may, optionally, include one or more corresponding engagement features (not shown) configured to interlock or otherwise engage with one or more of the engagement features 1422 of the port 1420. In these variations, the corresponding engagement features may align and interact through the elastomer layers of the elongate body 1402 (i.e., with the elastomer layers and braided layer positioned therebetween) to facilitate sealing. An engagement feature of the port and/or crimp may also resist tensile forces applied to the port 1420 or an end cap, thereby helping maintain the position of the port 1420 (e.g., the port body 1421) within the lumen 1408 under pressure.

The port 1420 may include a port lumen 1426 in fluid communication with the elongate body lumen 1403. In some variations, the port may comprise a port body 1421 and a flange 1423. The flange 1424 may be configured to receive the fluid (e.g., hydraulic) input pipe 1430. The port body 1421 may be sized to fit within the elongate body lumen 1408 and may include the engagement feature 1422. A locking feature 1423 may be positioned proximal to the engagement feature 1422. In some variations, the locking feature 1423 may be located outside the elongate body lumen 1408. The locking feature 1423 may include one or more protrusions, rings, or other raised structures configured to provide a mechanical anchor for the crimp 1430.

The port body 1421 may comprise a port body lumen having a first diameter, while the flange 1424 may comprise a flange lumen having a second, different (e.g., larger) diameter. The flange lumen, for instance, may comprise an inner diameter about equal to an outer diameter of the fluid input pipe or greater. Put differently, the flange may be configured to receive a distal end of the fluid input pipe in the flange lumen. This configuration may facilitate efficient fluid transfer to the artificial muscle 1400 while also providing support for the fluid input pipe 1440. Furthermore, in some variations, the end cap 1420 may comprise one or more anchors or fasteners to couple an end of an artificial muscle 1400 to a treatment arm and/or a cable for actuating a treatment arm.

Referring again to FIG. 13B, the artificial muscle 1300 may further include an end cap 1350 positioned at least partially within the elongate body lumen 1308. Similarly to the port 1320, the end cap 1350 may comprise an engagement feature and a locking feature, referring to herein as a cap engagement feature 1352 and a cap locking feature 1354. These features may function similarly to those of the port 1320 to seal the elongate body lumen on the opposing end. The cap engagement feature 1352 and cap locking feature 1354 may comprise any one or more of the features described with respect to the engagement feature 1322 and locking feature 1324 of the port. The end cap 1350 may be solid to completely seal one end of the elongate body lumen 1303. In certain variations, the end cap 1330 may be positioned at a first end of the elongate body 1302 and the port 1310 may be positioned at a second, opposite end. Furthermore, in some variations, the end cap 1350 may comprise one or more anchors or fasteners to couple an end of an artificial muscle 1300 to a treatment arm and/or a cable for actuating a treatment arm.

Returning to FIG. 14, the crimp 1430 may be configured to secure and seal the interface between the elongate body 1402 and the port 1420. The crimp 1430 may be positioned around the locking feature 1423 of the port 1420 and may compress a portion of the elongate body 1402 against the engagement feature 1422, thereby fixedly attaching (e.g., anchoring) the elongate body 1402 to the port body 1421. An end of the crimp 1430 may be received on a surface of the port body 1421 between the locking feature 1423 and the flange 1424. In some variations, the crimp 1430 may further secure the elongate body 1402 to the locking feature 1423 to ensure the elongate body lumen 1408 remains fluid-tight during actuation. The locking feature 1423 may include one or more rounded edges to reduce stress on the elastomer layers 1403, 1404 and/or the braided layer 1405 that may be compressed against the locking feature by the crimp 1430.

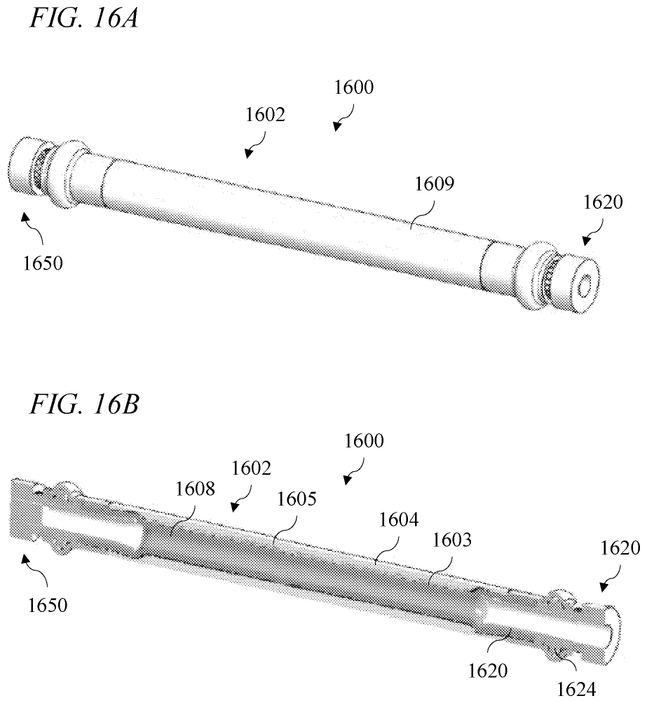

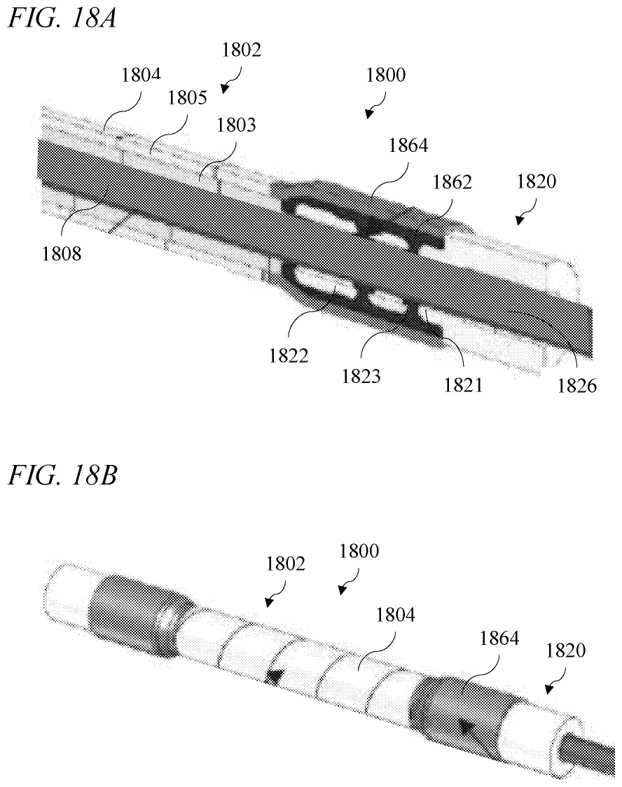

FIG. 15 depicts a cross-sectional view of an end portion of an artificial muscle 1500 including a port 1520 comprising a flared port body 1521. A port may generally have a cylindrical shape. As shown in FIG. 15, a portion of the port body 1521 positioned within the elongate body lumen 1508 may increase in diameter along its length. In this manner, at least a portion of the port body 1521 (e.g., distal portion) may have a conical shape. In some variations, it may be advantageous to utilize a port 1520 with a body 1521 that flares radially outward at a distal end thereof as this may improve retention of the port within the elongate body lumen 1508 when the artificial muscle 1500 is pressurized. In some variations, as shown in FIG. 17, the diameter increase (i.e., the flare) of the port body may be confined to a distal portion of the port body. For example, a proximal portion corresponding to about ¼, about ⅓, about ½, about ⅔, or about ¾ of the length of the port body may have an approximately uniform (e.g., consistent along the length) diameter. Thus, in certain variations, only a distal portion of port body corresponding to about ¾, about ⅔, about ½, about ⅓, or about ¼ of the length of the port body may increase in diameter.

FIG. 15 further depicts a crimp 1530 configured to secure the elongate body 1502 to the port 1520. In some variations, as shown in FIG. 14, the crimp may be deformed onto an outer surface of a portion of the elongate body 1402 and an outer surface of the port 1420. For example, the crimp may be die-pressed around an end portion of the elongate body and the locking feature of the port. In other variations, as shown in FIG. 15, the crimp 1530 may be preformed (e.g., machined) to fit the outer surface of the elongate body 1502 and the outer surface of the port (e.g., a taper of port body, a locking feature of the port, an engagement feature of the port). For example, the crimp 1530 may have an engagement feature similar to those described with respect to the port and end cap, that may receive a corresponding crimp engagement feature of the port. As another example, the crimp 1530 may have a locking feature such as a recess, that may receive a corresponding locking feature (e.g., protrusion) of the port. Furthermore, the crimp 1530 may generally be configured to fit the shape of the port body 1521. For example, as shown in FIG. 15, the crimp 1530 may comprise a conical lumen to fit a tapered port body 1521.

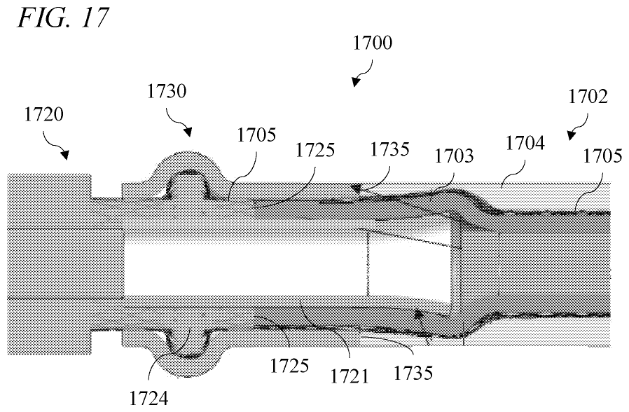

FIG. 16A depicts a perspective view of another exemplary artificial muscle 1600, and FIG. 16B depicts a perspective cross-sectional view of the artificial muscle 1600 shown in FIG. 16A. The artificial muscle 1600 may comprise an elongate body 1602 including a first elastomer layer 1603, a second elastomer layer 1604, and a braided layer 1605 positioned between the two elastomer layers. The elastomer layers 1603, 1604 may be molded over a mandrel (not show) (e.g., to preserve an elongate body lumen 1608), the braided layer 1605, a port 1620, and/or an end cap 1650 to create an integrated structure. In some variations, one or both of the first elastomer layer 1603 and/or the second elastomer layer 1604 may comprise an uncured silicone. This molding process may improve sealing around the internal components and may reduce defects at the interface between the elastomer layers and the braided layer 1605.

To seal the artificial muscle 1600, a portion of the elongate body 1602 may be compressed between the body 1621 of the port 1620 and a crimp 1630. As shown in FIG. 16B, the compressed portion may include the first elastomer layer 1603 and the braided layer 1605. In other variations, such as the exemplary artificial muscle 1700 shown in FIG. 17, the compressed portion may consist solely of the braided layer. One or more of the elastomer layers 1603, 1604 and/or the port 1620 may overlap a locking feature 1624 of the port. In certain manufacturing methods, an elastomer layer may be molded around a mandrel to form the elongate body lumen 1608. The first elastomer layer 1603 may be positioned over and around the locking feature 1624 to improve sealing.

In some variations, as shown in FIG. 16, the elongate body 1602 of an artificial muscle may further comprise a coating 1609 configured to reduce vapor permeability. For example, the artificial muscle may include coating 1690 comprising a polyimide tube coated in stainless steel and bonded using a gap-filling epoxy adhesive. Such a coating may reduce vapor transmission while maintaining low bending stiffness.

FIG. 17 depicts a cross-sectional view of an end portion of an exemplary artificial muscle 1700 including an elongate body 1702 comprising first and second elastomer layers 1703, 1704 and a braided layer 1705, a flared port 1720, and a crimp 1730. In the variation shown in FIG. 17, the braided layer 1705 may be the only layer of the elongate body 1702 that overlaps a locking feature 1724 of the port 1720. Positioning the braided layer 1705 over the locking feature 1724 may mechanically anchor the braided layer 1705 at the end of the artificial muscle 1700. Anchoring the braided layer 1705 at both ends (e.g., over and/or around the port and the end cap) may allow the braid to more effectively constrain radial expansion and control axial contraction of the artificial muscle during pressurization. The port body 1721 of the port 1720 may increase in diameter in a distal direction, thereby providing additional surface area for engaging the elongate body 1702 and improving retention of the port 1720 within the elongate body lumen 1708 when the latter is pressurized. As described previously herein, the increase in diameter may be confined to a distal portion of the port body 1721.

The locking feature 1724 may be configured to provide one or more flat surfaces 1725 that support a first elastomer layer (e.g., layer 1703) when the elastomer layer is molded to form a portion of the artificial muscle 1700. The locking feature 1724 may further comprise a protrusion configured, in combination with the crimp 1730, secure at least the braided layer 1705 to the port. In addition to its sealing function, the crimp 1730 may provide a second flat surface 1735 that supports a second elastomer layer (e.g., layer 1704) during molding. In the variation of FIG. 17, the crimp 1730 may be positioned only around the first elastomer layer 1703 and the braided layer 1705 and not the second elastomer layer 1704. The crimp 1730 may further have a thickness approximately equal to the thickness of the second elastomer layer 1704 so that an outer surface of the crimp 1730 may be substantially flush with an outer surface of the elongate body 1702.

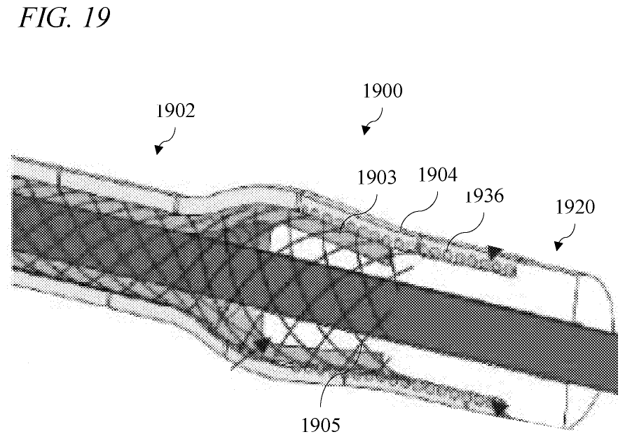

FIG. 18A depicts a cross-sectional view of another exemplary artificial muscle 1800 including an elastomer layer coupled to (e.g., molded to) a port 1820, and FIG. 18B depicts a perspective view of the artificial muscle 1800 shown in FIG. 18A. The artificial muscle 1800 may comprise an elongate body 1802 including a first elastomer layer 1803, a second elastomer layer 1804, and a braided layer 1805 positioned between the elastomer layers 1803, 1804. The elongate body 1802 may further include an elongate body lumen 1808 configured to receive a fluid to actuate the muscle. A port 1820 may be positioned at least partially within the elongate body lumen 1808 and may be secured to the elongate body 1802 via one or more port elastomer layers 1862, 1864 of the artificial muscle. In this variation, the artificial muscle 1800 may not include a crimp. Instead, one or more port elastomer layers 1862, 1864 may be configured to provide sealing, mechanical anchoring, and/or structural reinforcement at the interface between the port 1820 and the elongate body 1802.

The artificial muscle 1800 may further comprise one or more port elastomer layers 1862, 1864 configured to seal the port 1820 to the elongate body 1802. The port elastomer layers 1862, 1864 may be configured to secure the port 1820 to the elongate body 1802, maintain a fluid-tight seal, and resist tensile forces applied to the port 1820, similarly to the crimp structures described herein. These port elastomer layers 1862, 1864 may be used in addition to, or as an alternative to, the crimp structures described elsewhere in this application. Put differently, in some variations, the artificial muscles described herein (e.g., any of the artificial muscles described with respect to FIGS. 13A-20B) may include one or more crimps, one or more port elastomer layers, or a combination of one or more crimps and one or more port elastomer layers. The port elastomer layers 1862, 1864 may comprise a low-elasticity elastomer to improve mechanical anchoring. When multiple port elastomer layers are provided, a portion of the braided layer 1805 may be positioned between adjacent port elastomer layers to secure the braided layer 1805 at an end of the artificial muscle 1800.

In configurations such as the one depicted in FIG. 18, which include molded port elastomer layers 1862, 1864, an engagement feature 1822 of the port 1820 may be configured to engage and interlock with the port elastomer layers. The engagement feature 1822 may comprise one or more openings 1823 formed through a wall of the port body 1821. Each opening 1823 may extend from an outer surface of the port body 1821 to the port lumen 1826. The openings 1823 may be configured to receive a portion of a port elastomer layer (e.g., layer 1862) as it is molded, cured, and/or wrapped around the port 1820, thereby securing the port 1820 to the elongate body 1802. In some variations, a width of at least one opening 1823 may increase toward the port lumen 1826 to improve retention of a molded port elastomer layer 1862 that fills the opening. This may help the port elastomer layers 1862, 1864 resist tensile loads applied to the port 1820 under pressure, maintaining the position of the port within the elongate-body lumen 1808.

Additionally or alternatively, as depicted in FIG. 18, the first and/or second elastomer layers 1803, 1804 may comprise a wrapped strip of elastomer. For example, the first elastomer layer 1803 may be formed by wrapping a strip of elastomer around a mandrel, and the second elastomer layer 1804 may be formed by wrapping a strip of elastomer around the combination of the first elastomer layer 1803 and the braided layer 1805.

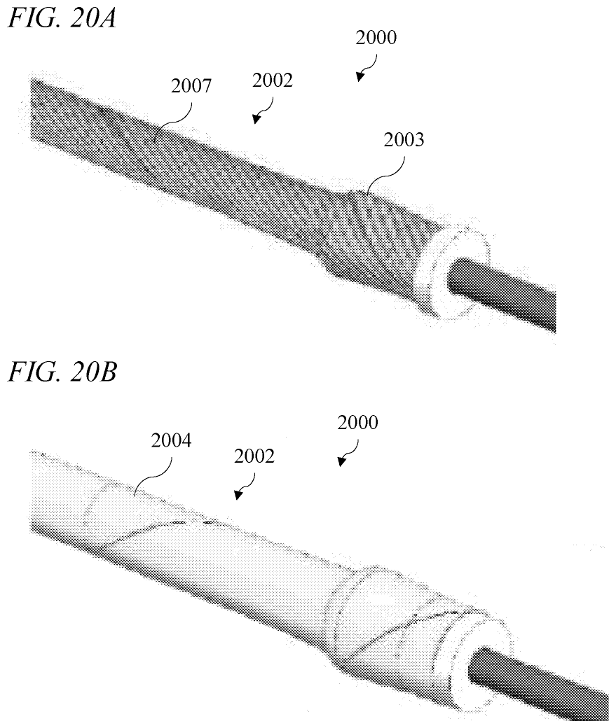

FIG. 19 depicts a cross-sectional view of yet another artificial muscle 1900 including a whip 1936 configured to secure a port 1920 to an elongate body 1902 of the artificial muscle 1900. The elongate body 1902 may comprise a first elastomer layer 1903, a second elastomer layer 1904, and a braided layer 1905 positioned between the elastomer layers, with an elongate body lumen 1908 extending therethrough. The artificial muscle 1900 may comprise a whip 1936 to, in addition to or as an alternative to a crimp, anchor the port 1920 within the elongate body lumen 1908. In certain variations, the whip 1936 may be wound around the first elastomer layer 1903, the braided layer 1905, and a portion of the port 1920 to secure the elongate body 1902 directly to the port. A second, outer elastomer layer 1904 may be positioned around the whip 1936 and the other layers of the elongate body 1902 to cover and further secure the whip. In other variations, the whip 1936 may be wound around all layers of the elongate body 1902 and may be configured to press one or more of those layers into contact with the port 1920 (e.g., into the port body or an engagement feature thereof) to improve sealing and mechanical retention of the port. The whip 1936 may comprise a string or wire, such as, for example, an uncured elastomer in a thin string.

In some variations, as described in more detail herein, an artificial muscle may be coupled to a base of a treatment manipulator. In particular, a proximal end of the artificial muscle including the port may be secured to the base. The base may be configured to secure the port to the elongate body in addition to, or as an alternative to, other securing structures described herein, such as crimps, whips, or molded elastomer layers. For example, the base may comprise one or more openings configured to receive a proximal portion of the artificial muscle, which may include the port and one or more layers of the elongate body. Within the opening, the base may compress the elongate body, and in particular the braided layer, against the port including one or more features thereof, such as an engagement feature or a locking feature. This compression may secure the port to the elongate body and may maintain a sealed interface between the elongate body and port during actuation of the artificial muscle

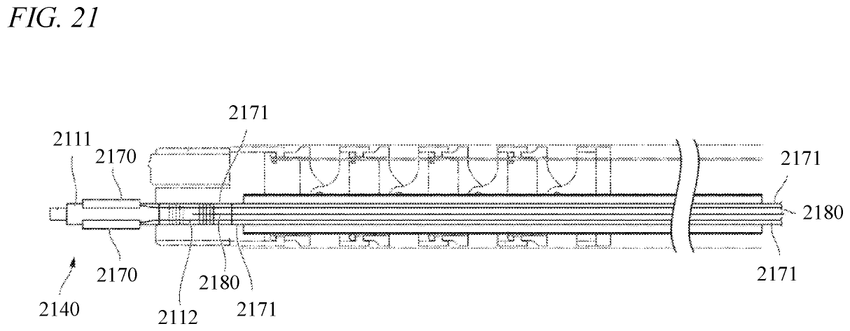

FIG. 20A depicts a perspective view of an artificial muscle 2000 including a first elastomer layer 2003, and FIG. 20B depicts a perspective view of the artificial muscle 2000 including a second elastomer layer 2004. In some variations, as shown in FIGS. 20A and 20B and as previously described with respect to FIGS. 18A and 18B, one or more elastomer layers of the elongate body 2002 may comprise a wrapped elastomer-strip construction. In such variations, the elastomer strips used to form the elastomer layers may include a plurality of fibers 2007. A longitudinal axis of each fiber 2007 may be aligned with a longitudinal axis of the elastomer strip prior to wrapping. When the strip is wrapped around a mandrel to form the first elastomer layer 2003, the fibers 2007 may be positioned at an angle relative to a longitudinal axis of the artificial muscle 2000. The second elastomer layer 2004 may be formed by wrapping a second elastomer strip around the first elastomer layer 2003 in an opposite winding direction, as shown in FIG. 20B, thereby orienting the fibers 2007 of the second strip at an opposing angle. When combined, the fiber orientations of the first and second elastomer layers 2003, 2004 may form a braided pattern between the layers. This braided pattern of fibers may control dimensional changes of the elongate body 2002 during actuation and may provide functional behavior similar to that of a braided layer used in other variations.

Turning back to FIGS. 13A and 13B, a fluid input pipe 1340 may be coupled to the artificial muscle 1300. The fluid input pipe 1340 may be configured to supply a fluid (e.g., a pressurized fluid) for actuating the artificial muscle 1300. The artificial muscle 1300 may be configured to contract (e.g., shorten) when fluid is supplied through the fluid input pipe 1340 to the elongate body lumen 1308. In some variations, the fluid input pipe 1340 may be coupled to (e.g., partially received in) a port 1320 of the artificial muscle 1300. To secure the input pipe 1340 to the artificial muscle 1300, the port 1320 may comprise a flange 1324 configured to receive a distal end of the fluid input pipe 1340. In certain variations, the fluid input pipe 1340 may extend from a proximal end of the artificial muscle 1300 through a treatment lumen of the insertion manipulator to a fluid source located outside the insertion manipulator.

The operation of the artificial muscles may be divided into a “nonlinear region” in which the amount of deformation relative to the amount of pressurization of the fluid is nonlinear, and a “linear region” in which the amount of change relative to the amount of pressurization of the fluid is linear (i.e., is actually linear or can be handled as approximately linear in terms of the control of the manipulator). When the fluid is pressurized from a non-pressurized state, the operation of the artificial muscle transitions to the linear region via the nonlinear region. Therefore, the artificial muscle is in the “initial state” when the fluid is compressed by pressurizing it until it transitions to the linear region, the responsiveness of the artificial muscle improves, and the controllability of one or more bending portion of the arm improves.

### Artificial Muscles Placement

The treatment manipulator may comprise one or more artificial muscles configured to actuate an arm of the treatment manipulator. These artificial muscles may be coupled to the arm in various locations and may be configured to bend, articulate, or otherwise move one or more bending portions of the arm to position and control a treatment tool at a target site. In general, the muscles may shorten or contract in response to pressurized fluid, and such contraction may apply forces to the arm that bend portions of the arm relative to one another. The artificial muscles may therefore function as actuators that drive movement of the treatment manipulator, enabling the operator to manipulate tissue and/or orient tools, and/perform therapeutic or diagnostic procedures.

The artificial muscles may be attached to the arm in a variety of configurations. Attachment points may be arranged to transmit contraction forces to specific bending portions of the arm, including a distal bending portion and/or a proximal bending portion comprising one or more shoulder joints. In some variations, attachment points of the arm may be configured to receive a proximal or distal end of the artificial muscles (e.g., an end cap, a port). Additionally or alternatively, ends of the muscles may be coupled to a point on the arm via one or more cables. For example, an end (e.g., an end cap, a port) of a muscle may be fixedly coupled to an arm directly or via cable at one or more joint rings, links, or similar elements of the bending portion.

Generally, a plurality of artificial muscles (e.g., two, three, four, or more muscles) may be arranged around the arm along a longitudinal axis of the arm. These muscles may be positioned symmetrically or asymmetrically around the arm to generate bending motions in multiple directions. Placement of the muscles around the circumference of the arm may be selected to provide independent control of bending portions of the arm. The muscles may be positioned at different axial locations along the arm and/or at more proximal locations on the treatment manipulator, such as along a portion of the treatment manipulator within the insertion manipulator. Such variations in muscle placement may allow different bending portions of the arm to be actuated independently or in combination. The following variations describe multiple examples of muscle placement that may be used to control movement of an arm of the treatment manipulator.

For example, as shown in FIG. 12, a plurality of artificial muscles 1270 may be coupled to a first, distal bending portion 1211 of an arm 1210 to manipulate that portion of the arm. A distal end of the arm 1210 and the distal ends of the artificial muscles 1270 may be connected to a distal attachment point of the treatment manipulator, such as a ring 1215. The proximal ends of the artificial muscles 1270 may be coupled to a proximal end of the proximal bending portion. When the artificial muscles 1270 actuate (e.g., contract, extend (e.g., relax), or bend), the first bending portion 1211 of the arm may bend in response. In the operating configuration of the treatment manipulator 1200 illustrated in FIG. 12, the first bending portion 1211 is disposed distally with respect to the distal end surface of the distal end portion 311. Accordingly, the artificial muscles 1270 may also be disposed distal to the distal end surface of the distal end portion 311 of the insertion manipulator.

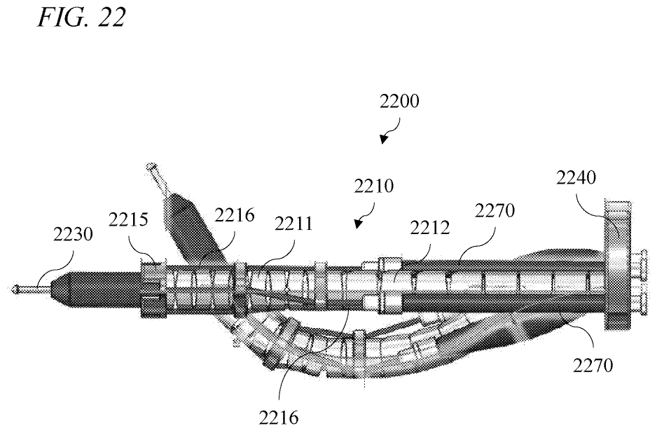

FIG. 21 depicts a cross-sectional view of an exemplary insertion manipulator 2110 and an exemplary treatment manipulator 2140 in which a plurality of artificial muscles 2170 is coupled to a first, distal bending portion 2111 of an arm 2110. The artificial muscles 2170 may be disposed distal to a second, proximal bending portion 2112. The first bending portion 2111 and the artificial muscles 2170 may be coupled directly or by a short length of cable (e.g., such as a length shorter than the artificial muscle itself) at their respective proximal ends and distal ends. When an artificial muscle 2170 actuates (e.g., contracts or relaxes), the first bending portion 2111 may bend away or towards that muscle. In this configuration, the artificial muscles 2170 may bend together with the first bending portion 2111. As illustrated in FIG. 21, the artificial muscles 2170 may be disposed entirely distal to a distal end portion 311 of the insertion manipulator while in use (e.g., in the operating configuration).

The first bending portion 2111 may be bendably driven by the artificial muscles 2170. The artificial muscles 2170 may bend the first bending portion 2111 with greater precision than a wire. Accordingly, the artificial muscles 2170 may be suitable for actuating the first bending portion 2111 in procedures in which fine or precise treatment operations are required. The artificial muscles 2170 may be attached directly to the first bending portion 2111 and may bend the first bending portion 2111 through its elongation and contraction. This direct coupling may permit accurate bending of the first bending portion 2111 without being influenced by the shape or configuration of structures located proximal to the first bending portion 2111. Each of the artificial muscles 2170 may be actuated via fluid provided by one or more fluid input pipes 2171.

In certain variations, while the first bending portion may be actuated by one or more artificial muscles as described above, additional bending portions of the arm (e.g., the second, proximal bending portion 2112) may be actuated using other actuators, including, for example, pull wires. A wire 2180 may be coupled to the second bending portion 2112. By pulling the wire 2180 from a proximal end, the second bending portion 2112 may be bent independently of the first bending portion.

Additionally or alternatively, the artificial muscles may be positioned on a second, proximal bending portion of the arm. The artificial muscles may be configured to bend the proximal bending portion as well as any associated shoulder joints. In these variations, the artificial muscles may not be disposed distal to a distal end of the second, proximal bending portion of the arm. Furthermore, the artificial muscles may be positioned proximal to a proximal end of a distal end portion of the insertion manipulator or a base of the treatment manipulator. That is, in some variations, the artificial muscles may be confined to the region of the second, proximal bending portion. This configuration may be referred to as shoulder-mounted muscles. The artificial muscles may be configured to move the second bending portion. For example, the artificial muscles may be configured to move the second bending portion into a notch or opening of the distal end portion of the insertion manipulator. Moreover, even though the artificial muscles may be coupled to the second, proximal bending portion, the muscles may also actuate the first, distal bending portion. Thus, in some variations, a plurality of artificial muscles may be configured to move both the proximal and distal bending portions of the arm of the treatment manipulator, as will be described in more detail herein.

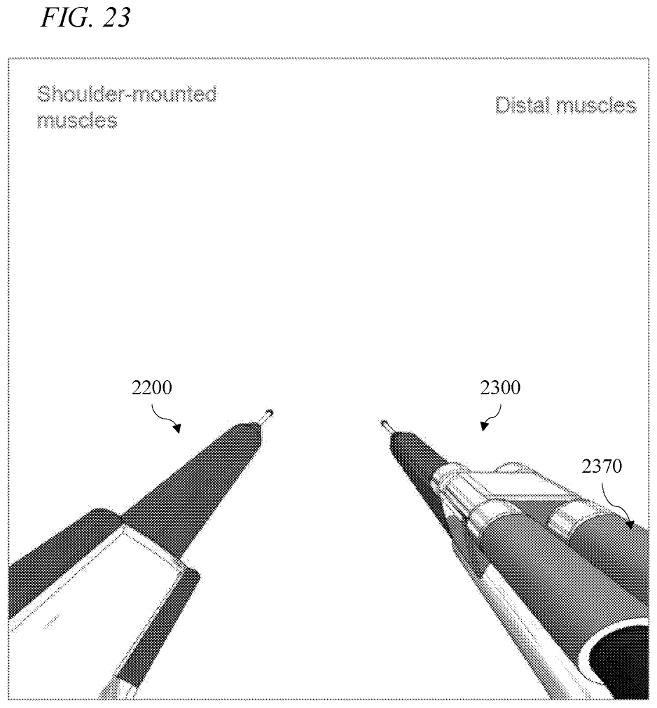

For example, FIG. 22 depicts an exemplary treatment manipulator 2200 including shoulder-mounted artificial muscles 2270. The treatment manipulator 2200 may comprise a base 2240, an arm 2210 including a first, distal bending portion 2211 and a second, proximal bending portion 2212, a plurality of artificial muscles 2270, and a treatment tool 2230. As shown in FIG. 22, a proximal end of the arm 2210 may be coupled to the base 2240. A distal end of the arm 2210 may be configured to move freely relative to the based in response input from the artificial muscles 2270.

The plurality of artificial muscles 2270 may be coupled to the second bending portion 2212 and may be configured to manipulate both the second bending portion 2212 and the first bending portion 2211. At their proximal ends, each of the artificial muscles 2270 may be coupled to the base 2240. In other variations, the proximal end of each artificial muscle may be coupled to a proximal end portion (e.g., a proximal ring) of the second bending portion 2212.

At a distal end, each artificial muscle 2270 may be coupled, directly or via a cable 2280, to a distal end portion (e.g., a distal ring 2215) of the second bending portion 2212. In some variations, as shown in FIG. 22, each artificial muscle 2270 may also be indirectly coupled to the first bending portion 2211 through the cable 2280. For example, the cable 2280 may be slidably coupled to one or more wire-guides 2216 positioned along the arm 2210 to actuate bending at one or more longitudinal locations. The cable 2280 may be fixedly coupled to a distal end of the first bending portion 2211 and to the plurality of artificial muscles 2270, enabling the muscles to actuate both bending portions of the arm in combination.

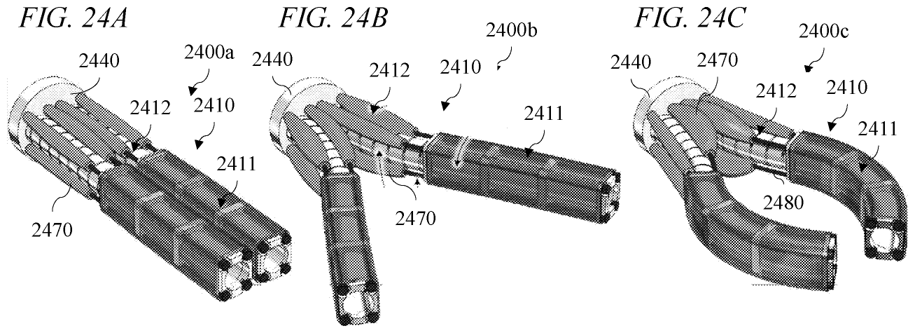

Advantageously, a treatment manipulator comprising artificial muscles in the shoulder-mounted configuration may provide an imaging unit of the endoscope system with improved visibility of one or more treatment tools. FIG. 23 depicts an illustrative variation of the field of view of an imaging unit observing the treatment manipulator 2200 of FIG. 22 and a treatment manipulator 2300 having artificial muscles 2370 positioned along the distal bending portion. As seen from the perspective of the imaging unit in FIG. 23, a treatment manipulator 2200 comprising shoulder-mounted artificial muscles (not visible) may occupy a reduced portion of the imaging unit's field of view, thereby improving visibility of a treatment tool positioned at or extending from the distal end of the arm. In some variations, the system may be configured such that the plurality of artificial muscles remains outside the field of view of a camera at the distal end of the treatment manipulator when a tool received within the treatment manipulator is in use.

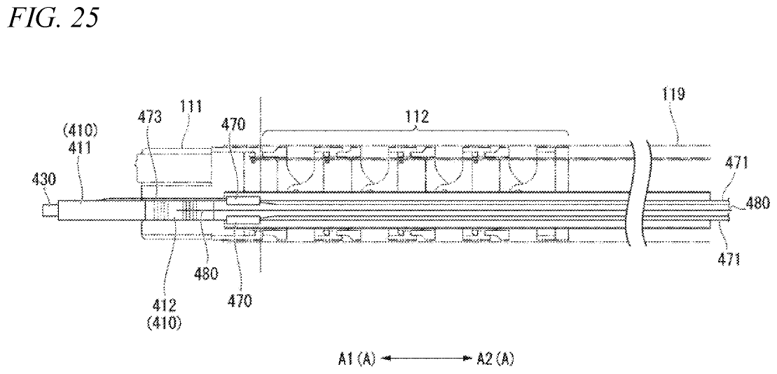

FIGS. 24A-24C depict an exemplary treatment manipulator in three configurations. The treatment manipulators 2400a-c may comprise a base 2440, an arm 2410 including a first, distal bending portion 2411 and a second, proximal bending portion 2412, and a plurality of artificial muscles 2470 arranged along the proximal bending portion. The plurality of artificial muscles 2470 may be coupled to the arm 2410 and may be configured to actuate one or both bending portions depending on their state of contraction. These figures illustrate how the treatment manipulator may transition from a delivery configuration to a deployment configuration and finally to an operating configuration during use.

FIG. 24A shows the treatment manipulator 2400A in a delivery configuration. In this configuration, the arm 2410 and the first and second bending portions 2411, 2412 may be straight or nearly straight and aligned along a longitudinal direction. Such alignment may reduce a transverse dimension of the treatment manipulator 2400A to facilitate insertion into a treatment lumen of an insertion manipulator. The artificial muscles 2470 may be relaxed in this configuration. Overall, the arms 2410 and artificial muscles 2470 may assume an elongated, low-profile shape suitable for delivery in this configuration.

FIG. 24B depicts the treatment manipulator 2400B in a deployment configuration. In this configuration, one or more of the artificial muscles 2470 may be at least partially contracted to bend the second, proximal bending portion 2412. This bending may also cause one or more shoulder joints (not shown) to articulate. The curvature of the proximal bending portion 2412 may position the arm 2410 to enhance the reach or visibility of the system. In some variations, the proximal bending portion 2412 may be locked to hold the curvature of this configuration. This configuration may allow the artificial muscles to manipulate the distal bending portion 2411 independent of the proximal bending portion.

FIG. 24C depicts the treatment manipulator 2400C in an operating configuration. In this configuration, the proximal bending portion 2412 may be configured to lock in a curved configuration shown in the deployment configuration of the treatment manipulator. Locking may be achieved, for example, by one or more vertebrae of the arm locking to one another or friction-based interfaces that resist movement of that portion of the arm. Locking the proximal bending portion 2412 may enable forces generated by the artificial muscles 2470 to be transferred efficiently to the distal bending portion 2411, such as through cables 2480 or direct couplings. Once locked, the proximal bending portion 2412 may act as a stable foundation that supports precise motion of the distal bending portion 2411 via actuation of the artificial muscle 2470.

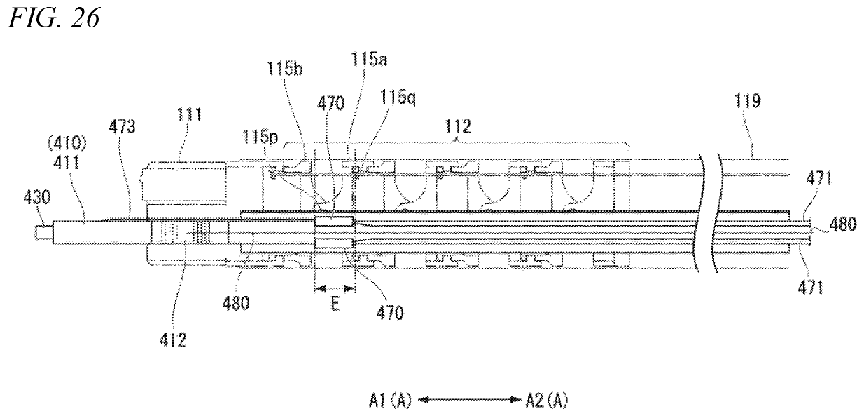

FIGS. 25-29 depict various other configurations of exemplary treatment manipulators with alternative artificial muscle positions. FIG. 25 depicts a cross-sectional view of an exemplary insertion manipulator and treatment manipulator in which one or more artificial muscles 470 are positioned proximal to a second, proximal bending portion 412 of an arm 411. A distal end wire 473 may couple the artificial muscle 470 to a distal end of the first, distal bending portion 411. The artificial muscle 470 may actuate the first bending portion 411 by pulling or relaxing the distal end wire 473, thereby moving the first bending portion 411 forward or rearward. As shown in FIG. 25, the artificial muscle 470 may be located within a distal end portion 111 of the insertion manipulator that does not bend. Accordingly, even when the bending portion 120 is bent during operation, the artificial muscle 470 may remain unbent and protected within the non-bending region of the distal end portion 111.

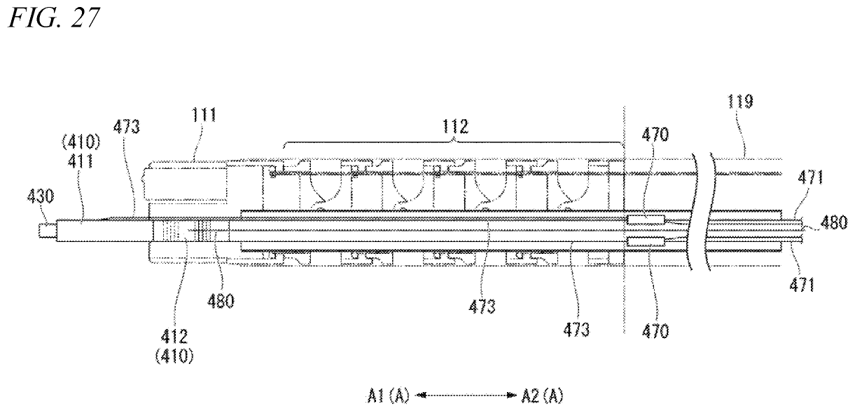

FIG. 26 depicts a cross-sectional view of an exemplary endoscope system in which an artificial muscle 470 is positioned within the bending portion 112 but disposed in a non-bending area E. The non-bending area E may be defined as a region between a first rotary pin and a second rotary pin. Because area E does not experience angular deflection during bending, the artificial muscle 470 positioned in this region may remain straight even when the bending portion 120 bends around the rotary pins. This configuration may allow the artificial muscle 470 to be positioned close to the bending portion while avoiding unwanted deformation during operation.

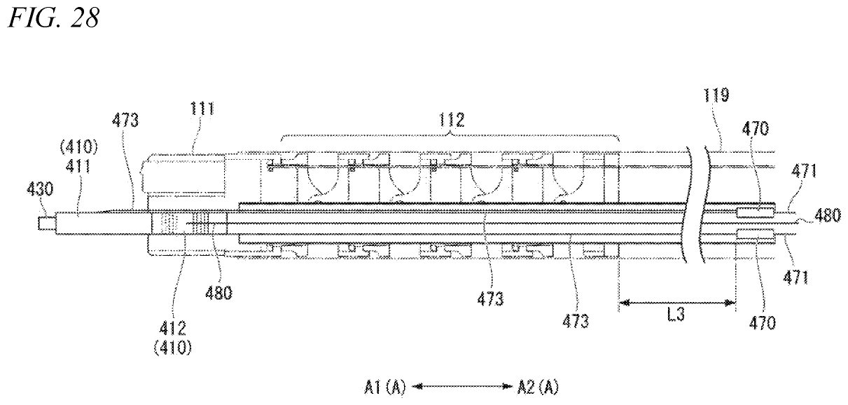

FIG. 27 depicts a cross-sectional view of an exemplary endoscope system in which an artificial muscle 470 is disposed within a distal portion of the flexible portion 119 near the bending portion 112. The flexible portion 119 may bend only slightly during operation. Accordingly, even when the bending portion 120 undergoes substantial bending, the artificial muscle 470 located within the flexible portion 119 may experience minimal bending or none at all. This placement may enable reliable actuation without requiring the artificial muscle to undergo the same curvature as the primary bending portion.

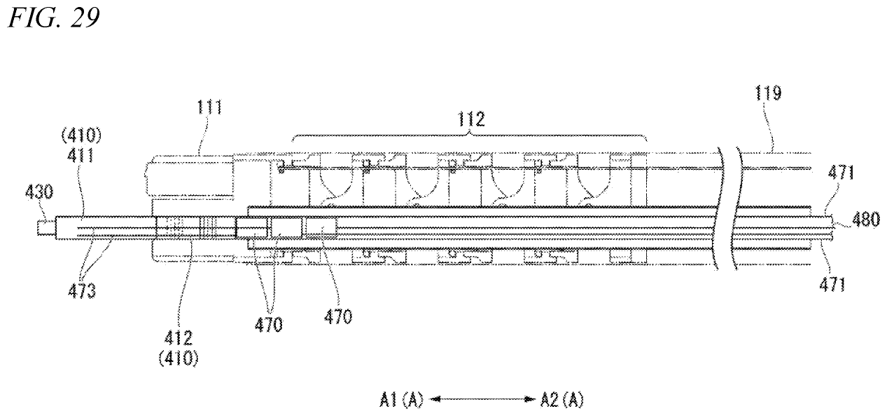

FIG. 28 depicts a cross-sectional view of an exemplary endoscope system including an artificial muscle 470 positioned within a proximal portion of a flexible portion 119 of the insertion manipulator. The artificial muscle 470 may be located at a distance L3 from the proximal end of the bending portion 112. The distance L3 may be selected to be greater than a forward or rearward movement distance of the treatment manipulator 400B during use. As a result, even when the treatment manipulator 400B translates distally or proximally during treatment, the artificial muscle 470 may not enter the bending portion 112. Thus, the artificial muscle 470 may remain in a region that does not bend, regardless of bending of the bending portion 120.

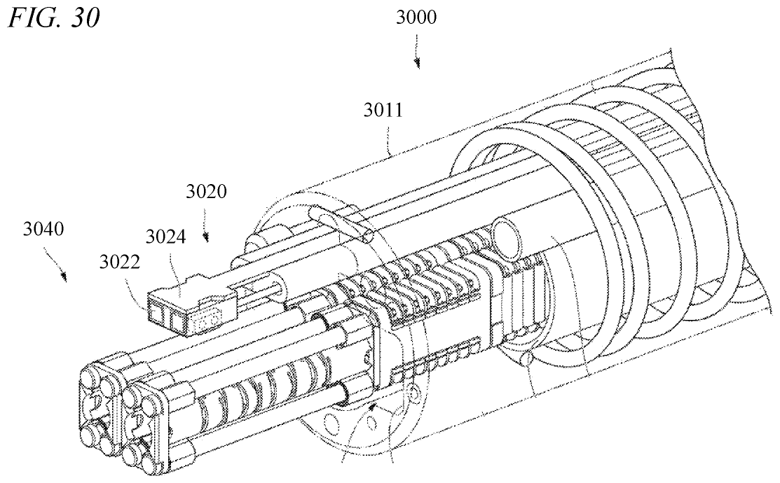

FIG. 29 depicts a cross-sectional view of an exemplary endoscope system in which a plurality of artificial muscles 470 is arranged in series along the axial direction A. The muscles 470 may be distributed so that one or more of the muscles are positioned within non-bending areas of the insertion manipulator. Arranging the muscles in the axial direction rather than in the radial direction may allow a reduced outer diameter of the treatment manipulator 400B. This configuration may also permit design flexibility in selecting which muscles actuate different bending portions depending on their locations along the axis.

### Scope

A scope may be provided to deliver visualization of both the target site and the treatment manipulator during use. The scope may be coupled to or positioned near a distal end portion of the insertion manipulator. The scope may be configured to extend or protrude distally to obtain an unobstructed view of the treatment region. In some variations, the scope may be bendable or steerable so that its viewing direction can be adjusted relative to the treatment manipulator.

The scope may comprise an imaging unit configured to capture images of tissue, the treatment manipulator, and/or treatment tools. The imaging or camera unit may include a housing, one or more lenses, and a camera. An illumination unit may also be included and may emit light toward the target region to enhance visibility during imaging. For example, the illumination unit may comprise one or more optical fibers. In some variations, the scope may further comprise an elongate body configured to be positioned within the treatment manipulator. The elongate body may comprise one or more cables or fibers configured pass signals between the imagining and/or illumination unit of the scope (e.g., the camera) and the control unit and/or operating units of the system. The imaging unit and illumination unit may operate together to provide real-time visualization.

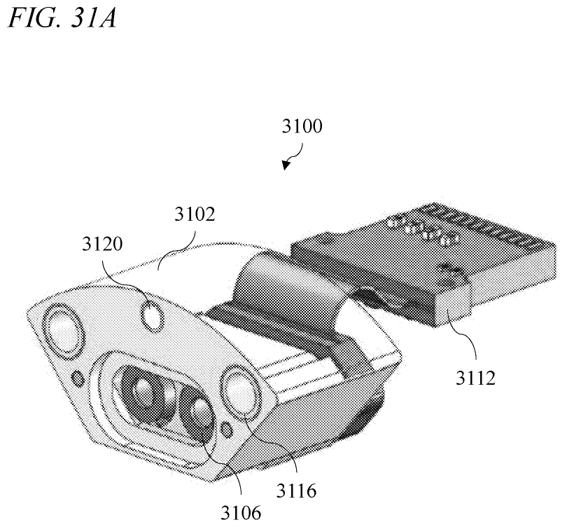

FIG. 30 depicts an exemplary endoscope system 3000 including a scope 3020. The scope 3020 may be coupled to the insertion manipulator 3010 such that it may protrude from the distal end portion of 3011 of the insertion manipulator 3010. In some variations, as will be described in more detail herein, the scope 3020 may be moveable (e.g., bendable, translatable). For example, the scope 3020 may be actuated by a scope-operation wire (not shown) to translate and/or change an angular orientation of the scope and, in particular, the imaging unit of the scope 3022. Generally, the scope may include an imaging unit 3022 and a support portion 3024 configured to support the imaging unit. The imaging unit 3022 may be positioned at a distal end of the support portion 3024 to capture images of the treatment manipulator, treatment tools, and/or the target site. In some variations, the scope 3320 may be configured to move axially relative to the insertion manipulator 3010 (e.g., advance and/or retract relative to the distal end portion 3011). In some variations, the scope 3320 may be further configured to bend relative to the insertion manipulator 3010 and the treatment manipulator 3040 (e.g., bend toward the treatment manipulator 3040). In some variations, the scope may be hermetically sealed to keep out contaminates out of scope (e.g., out of the scope housing).

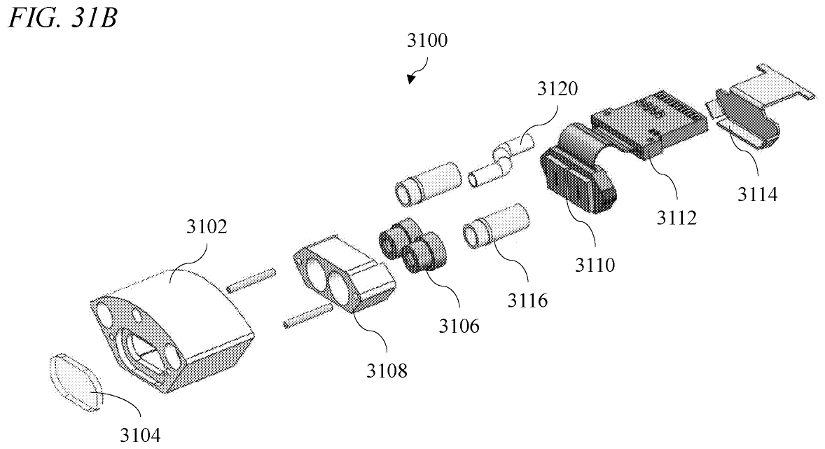

FIG. 31A depicts a perspective view of an exemplary imaging unit 3100, and FIG. 31B depicts an exploded view of the imaging unit 3100 of FIG. 31A. The imaging unit 3100 may comprise a housing 3102 configured to contain and protect a camera assembly. A window 3104 may be coupled to a front portion of the housing 3102 and may allow the camera 3110 to capture images through the window during operation. The housing 3102 may further be configured to hold one or more optical lenses 3106. In some variations, the optical lenses 3106 may be supported within the housing 3102 by a lens chassis 3108 to ensure proper optical alignment relative to the camera 3110.

The camera 3110 may be positioned within the housing 3102 proximal to the window 3104 and the optical lenses 3106. In certain variations, the camera 3110 may comprise an image-sensor printed circuit board (PCB) 3112 and a flexure 3114 configured to support or suspend the PCB within the housing 3102. The imaging unit 3100 may further comprise one or more optical-fiber lenses 3116 configured to focus illumination transmitted through one or more illumination fibers 3118 of the scope. The optical-fiber lens 3116 may be coupled to and held by the housing 3102 separately from the camera 3110.

Optionally, the imaging unit 3100 may comprise a hose 3120 configured to deliver cleaning fluid toward the window 3104 and/or to evacuate debris away from the window to maintain visibility of the camera. In some variations, the imaging unit 3100, and in particular the housing 3102, may be hermetically sealed to protect the internal components from contamination during endoscopic operation.

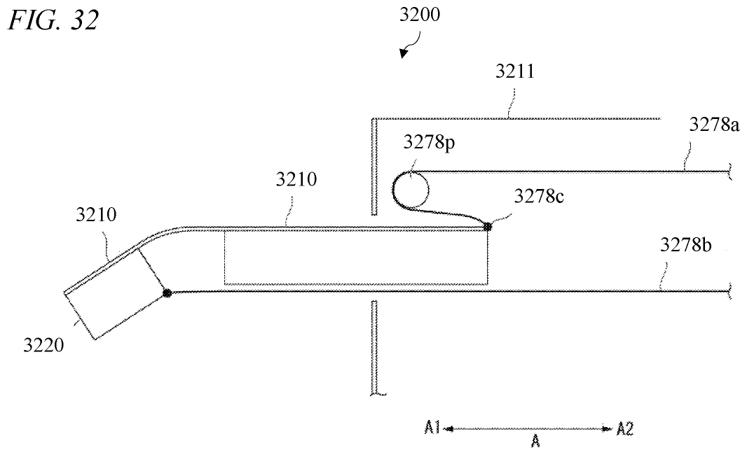

In some variations, the scope may be bendable to adjust its viewing direction toward a target site or toward a portion of the treatment manipulator. FIG. 32 depicts an exemplary scope 3200 configured to bend relative to a longitudinal axis of a camera lumen of the insertion manipulator. In some variations, the scope 3200 may include one or more bendable sections that allow the imaging unit to deflect away from or toward the longitudinal axis of the scope. The scope 3200 may be actuated by one or more pull wires 3278, as described herein, or alternatively by another actuator positioned within the insertion manipulator.

The scope 3200 may include a support portion 3220 that houses or supports the imaging unit. A leaf spring 3210 may be positioned along one side of the support portion 3220 and may be configured to bias the scope 3200 into a straight configuration when the pull wires are relaxed. The leaf spring 3210 may maintain the imaging unit in a stable orientation along the longitudinal direction when the scope is in a neutral or undriven state.

The scope 3200 may include a plurality of pull wires 3278a, 3278b configured to bend and/or translate the scope. For example, a first pull wire 3278a may be routed along a portion of the support structure and may be redirected by a pulley 3278p within the distal end of the insertion manipulator. A second pull wire 3278b may be connected to an underside of the imaging unit. The first and second pull wires 3278a, 3278b may be configured to operate independently or in combination.

When the first pull wire 3278a is pulled proximally, the scope 3200 may advance relative to the distal end portion of the insertion manipulator 3211. When the second pull wire 3278b is pulled proximally, the scope 3200 may retract relative to the distal end 3211. These axial movements may adjust the position of the imaging unit within the distal end region and may allow the system to optimize viewing distance and/or avoid occlusion by the treatment manipulator.

When both pull wires 3278a and 3278b are tensioned simultaneously, the pull force on the second pull wire 3278b may exceed the restoring force of the leaf spring 3210, causing the leaf spring to deflect. This may result in downward bending of the scope 3200 relative to the longitudinal axis, allowing the viewing direction of the imaging unit to be aimed toward a target site, a distal bending portion of the treatment manipulator, and/or a tool.

In this configuration, the pull-wire system may be configured to provide both forward and backward axial translation of the scope 3200 as well as angular bending of the imaging unit. Accordingly, the distal end portion of the insertion manipulator may be simplified and made thinner because a single pair of pull wires may achieve multiple degrees of motion.

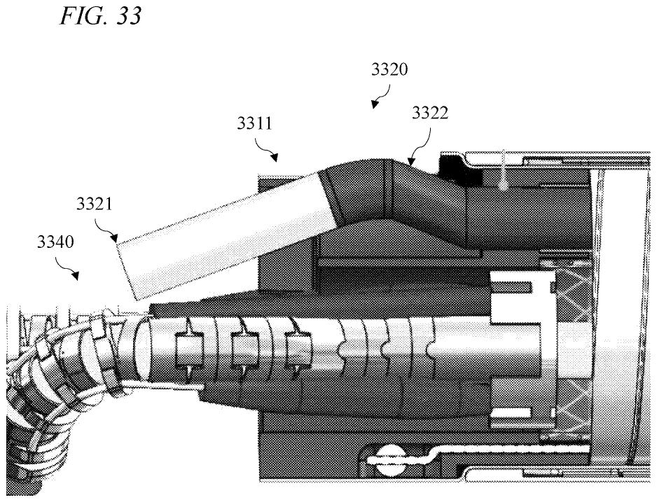

FIG. 33 depicts a side view of an exemplary adjustable scope 3320 showing the scope bent toward a treatment manipulator 3340. The scope 3320 may be configured to bend downward to align a viewing direction toward a treatment tool and/or a portion of the treatment manipulator. In some variations, as shown in FIG. 33, the scope 3320 may include more than one bend point. For example, the scope may comprise two or more bendable portions arranged in series along a distal end of the elongate body 3322 of the scope. This configuration, referred to as a “nod” configuration, may allow the scope 3320 to bend at multiple independent angles relative to the longitudinal axis. In some variations, the distal end portion 3311 of the insertion manipulator may comprise a recess or notch extending though a side of the insertion section to accommodate the bendable portion of the scope. Each bend point may be formed by a living hinge, pinned joint, or other compliant mechanism.

The bend points of the scope 3320 may be actuated by one or more pull wires (not shown). A first bend point may be positioned near a distal tip of the imaging unit 3300, and a second bend point may be positioned proximally. A scope comprising two or more bend points may achieve greater net angular deflection relative to the longitudinal axis and may therefore provide improved viewing of the treatment manipulator and treatment tools. A scope 3320 comprising one or more bend points may be configured to allow angular adjustment of the imaging unit housing by about 45 degrees or less, about 20 degrees or less, about 0 degrees to about 45 degrees, about 5 degrees to about 30 degrees, or about 10 degrees to about 20 degrees, including all subranges and values in any of the foregoing. A distal end of the imaging unit housing 3321 may additionally deflect by about 0 mm to about 10 mm, including all subranges and values therebetween, such as about 5 mm or less, providing improved visualization of the target site. In some variations, the scope 3320 may further include one or more actuators.

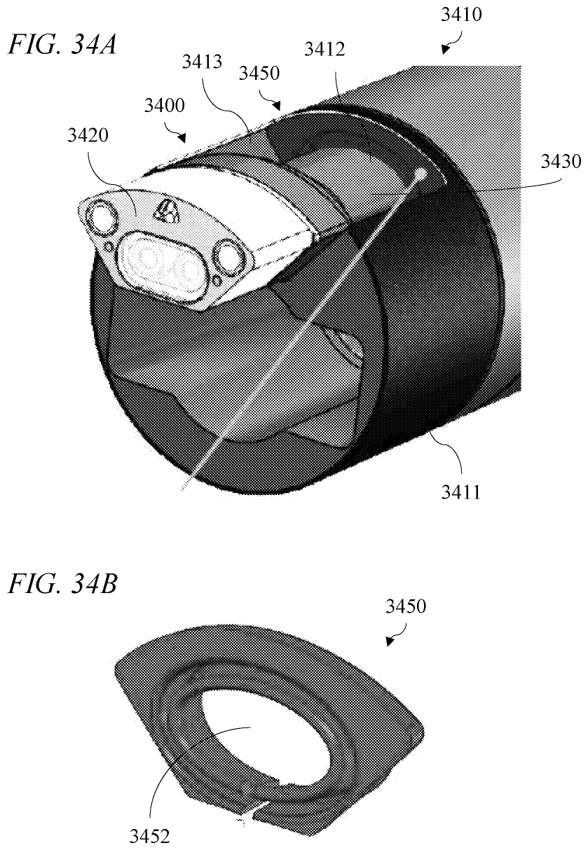

FIG. 34A depicts an exemplary endoscope system including a slidable scope 3400, and insertion manipulator 3410, and a camera lumen seal 3450. An insertion manipulator 3410 may comprise a distal end portion 3411 having a camera lumen 3412 and a recess 3413 formed at a distal end of the camera lumen. The recess 3413 may have a larger cross-sectional dimension than the cross-sectional dimension of the camera lumen 3412 and may be shaped to receive an imaging unit 3420 of the scope 3400. The imaging unit 3420 and recess 3413 may have corresponding sizes and shapes (e.g., both may have a circular, elliptical, rectangular, trapezoidal, or the like cross-sectional shape; a length of the imaging unit may be about equal to or less than a length) such that the imaging unit 3420 may be received within the recess 3173. The imaging unit 3420 and the recess 3413 may be positioned so that a shorter side or reduced dimension of the imaging unit 3420 is aligned with a central axis of the insertion section to minimize obstruction of other lumens. The scope 3400 may further comprise an elongate body 3430 coupled to the imaging unit 3420, the elongate body 3430 being slidably received within the camera lumen 3412 to adjust the axial position of the scope relative to the distal end portion 3411.

FIG. 34B illustrates the camera lumen seal 3450. The seal 3450 may be positioned at or near a distal opening of the camera lumen 3412 and may be configured to receive the elongate body 3430 of the scope 3400 through an opening 3452 therein. The seal 3450 may prevent contaminants, debris, or bodily fluids from entering the camera lumen 3412 as the scope 3400 is translated. In some variations, the seal 3450 may further stabilize the elongate body 3430 during translation by limiting radial play within the camera lumen 3412, thereby maintaining alignment of the imaging unit 3420 within the recess 3413 during use. The seal may have a shape corresponding to the shape of the recess 3413 and/or the imaging unit 3420.

In certain variations, the insertion section 3410 may be configured to receive the scope 3400 from the distal end. For example, a proximal end of the elongate body 3430 may be configured to be inserted through the opening 3452 in the seal 3450 and through a distal opening of the camera lumen 3412. The elongate body 3430 may then be further advanced proximally through the camera lumen 3412 until the imaging unit 3420 is positioned within the recess 3413. In this manner, the elongate body 3430 of the scope may be received within the insertion section 3410 before the imaging unit 3420 is positioned within the recess 3413. This configuration may allow the elongate body 3430 to be first seated and guided within the lumen, after which the imaging unit 3420 may be received and stabilized within the recess 3413. This configuration may further be advantageous for reducing contamination of the scope.

Also described herein are methods of using endoscopic surgical systems. In one variation, a method may include inserting a scope into a camera lumen of an insertion manipulator from a distal end of the insertion manipulator such that an elongate body of the scope becomes positioned within the camera lumen. The method may further include introducing the insertion manipulator into a patient to perform a surgical procedure. During or after the procedure, the scope may be withdrawn from the distal end of the camera lumen by sliding the elongate body distally and out of the camera lumen. This removal may help reduce or prevent contamination of the camera lumen and may simplify cleaning, sterilization, or reprocessing steps.

In some variations, a housing of the scope may be received within a recess at the distal end of the camera lumen when the scope is inserted. The method may further comprise adjusting the axial position of the scope by sliding the scope within the camera lumen during the procedure to optimize visualization of the target site or a treatment manipulator. A scope used in such methods may be hermetically sealed to protect internal components when inserted, withdrawn, or repositioned within the camera lumen. This sealing may further assist in maintaining optical clarity and durability during repeated clinical use.

While certain variations are described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive variations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations described herein. It is, therefore, to be understood that the foregoing variations are presented by way of example only and that, within the scope of the appended claims and equivalents thereto; inventive variations may be practiced otherwise than as specifically described and claimed. Inventive variations of the present disclosure are directed to each individual feature and/or method described herein. In addition, any combination of two or more such features and/or methods, if such features and/or methods are not mutually inconsistent, is included within the inventive scope of the present disclosure.

1. An artificial muscle, comprising:
a tubular elongate body comprising a first elastomer layer forming an elongate body lumen, a second elastomer layer around the first elastomer layer, and a braided layer between the first and the second elastomer layers;
a port positioned at least partially within the elongate body lumen and comprising an engagement feature and a locking feature; and
a crimp coupled to a proximal portion of the elongate body and the port, wherein the crimp is configured to seal the elongate body to the port via the engagement feature and the locking feature.

2. The artificial muscle of claim 1, wherein the elongate body lumen is configured to receive a fluid, the elongate body further configured to contract when the fluid is pressurized.

3. (canceled)

4. The artificial muscle of claim 2, wherein the braided layer constrains longitudinal expansion of the tubular elongate body.

5. The artificial muscle of claim 1, wherein the engagement feature comprises one or more grooves or threads.

6. (canceled)

7. The artificial muscle of claim 1, wherein the engagement feature is a first engagement feature and the crimp comprises a second engagement feature configured to interlock with the first engagement feature through the elongate body.

8. (canceled)

9. The artificial muscle of claim 1, wherein the port comprises a port lumen in fluid communication with the elongate body lumen, a port body, and a flange.

10. (canceled)

11. The artificial muscle of claim 9, wherein the port body is configured to be positioned within the elongate body lumen.

12. The artificial muscle of claim 9, wherein the port body comprises the engagement feature.

13. The artificial muscle of claim 12, wherein the port body comprises the locking feature, wherein the locking feature is proximal to the engagement feature.

14. The artificial muscle of claim 9, wherein the port body comprises a port body lumen of a first diameter and the flange comprise a flange lumen of a second, different diameter.

15. The artificial muscle of claim 9, wherein the crimp is received on a surface of the port body between the locking feature and the flange.

16.-18. (canceled)

19. The artificial muscle of claim 1, wherein the crimp is positioned around the locking feature of the port.

20. The artificial muscle of claim 1, wherein the crimp is configured to compress a portion of the elongate body against the engagement feature and to secure the elongate body to the locking feature.

21. The artificial muscle of claim 1, wherein the locking feature is outside of the elongate body lumen.

22. The artificial muscle of claim 1, wherein the locking feature comprises a protrusion.

23. (canceled)

24. The artificial muscle of claim 1, wherein a portion of the elongate body is compressed between the engagement feature of the port and the crimp.

25. The artificial muscle of claim 24, wherein the portion comprises the first elastomer layer and the braided layer.

26.-27. (canceled)

28. The artificial muscle of claim 1, wherein of the braided layer is positioned around the locking feature.

29. The artificial muscle of claim 1, wherein a portion of the port within the elongate body lumen increases in diameter along a length of the port within the elongate body.

30. (canceled)

31. An artificial muscle, comprising:
a tubular elongate body comprising a first elastomer layer forming an elongate body lumen, a second elastomer layer around the first elastomer layer, and a braided layer between the first and the second elastomer layers;
a port positioned at least partially within the elongate body lumen, the port comprising one or more grooves to engage the elongate body and a circumferential protrusion to lock the port to the elongate body; and
a crimp coupled to a proximal portion of the elongate body and the port, wherein the crimp is configured to seal the elongate body to the port via the one or more grooves and the circumferential protrusion.

32.-59. (canceled)

60. An endoscopic surgical system, comprising:
an insertion manipulator comprising a distal end; and
a treatment manipulator at least partially positioned in the distal end of the insertion manipulator, the treatment manipulator comprising:
a base;
an arm comprising a proximal end coupled to the base; and
a plurality of the artificial muscles of claim 1, wherein a proximal end of each muscle is coupled to the base and a distal end of each muscle is coupled to a distal end of the arm.
